SEQUENCE LISTING (30 sequences)

SEQ ID NO: 1 - DNA, 25 bp, synthetic construct
  misc_feature 1..25: note = Description of sequence: EGE-YQH-075-B-5'loxP-F (Hs3st1-5F)
  source 1..25: mol_type = other DNA
ggacggaatcataggcttcttgcct

SEQ ID NO: 2 - DNA, 25 bp, synthetic construct
  misc_feature 1..25: note = Description of sequence: EGE-YQH-075-B-5'loxP-R (Hs3st41-5R)
  source 1..25: mol_type = other DNA
cctcacctccctcaaacgctctatt

SEQ ID NO: 3 - DNA, 26 bp, synthetic construct
  misc_feature 1..26: note = Description of sequence: EGE-YQH-075-B-3'loxP-F (Hs3st1-3F)
  source 1..26: mol_type = other DNA
ggctggtcacaaaagccagttctaag

SEQ ID NO: 4 - DNA, 26 bp, synthetic construct
  misc_feature 1..26: note = Description of sequence: EGE-YQH-075-B-3'loxP-R (Hs3st1-3R)
  source 1..26: mol_type = other DNA
gcaccaggaaaaacatgtcaggtcaa

SEQ ID NO: 5 - DNA, 25 bp, synthetic construct
  misc_feature 1..25: note = Description of sequence: EGE-YQH-075-B-5'loxP-F (Hs3st1-5F)
  source 1..25: mol_type = other DNA
ggacggaatcataggcttcttgcct

SEQ ID NO: 6 - DNA, 25 bp, synthetic construct
  misc_feature 1..25: note = Description of sequence: EGE-YQH-075-B-5'loxP-R(Hs3st41-5R)
  source 1..25: mol_type = other DNA
cctcacctccctcaaacgctctatt

SEQ ID NO: 7 - DNA, 25 bp, synthetic construct
  misc_feature 1..25: note = Description of sequence: EGE-YQH-075-B-5'loxP-F (Hs3st1-5F)
  source 1..25: mol_type = other DNA
ggacggaatcataggcttcttgcct

SEQ ID NO: 8 - DNA, 26 bp, synthetic construct
  misc_feature 1..26: note = Description of sequence: EGE-YQH-075-B-3'loxP-R(Hs3st41-5R)
  source 1..26: mol_type = other DNA
gcaccaggaaaaacatgtcaggtcaa

SEQ ID NO: 9 - DNA, 25 bp, synthetic construct
  misc_feature 1..25: note = Description of sequence: EGE-YMX-006-A-5'loxP-F2 (Hs3st4-5F)
  source 1..25: mol_type = other DNA
aatcaggggtcagaaaaccgcctta

SEQ ID NO: 10 - DNA, 25 bp, synthetic construct
  misc_feature 1..25: note = Description of sequence: EGE-YMX-006-A-5'loxP-R2 (Hs3st4-5R)
  source 1..25: mol_type = other DNA
ggctgtgggaaaaagcccaaatgat

SEQ ID NO: 11 - DNA, 25 bp, synthetic construct
  misc_feature 1..25: note = Description of sequence: EGE-YMX-006-A-3'loxP-F2 (Hs3st4-3F)
  source 1..25: mol_type = other DNA
gtcacccaatgaggcactggctaaa

SEQ ID NO: 12 - DNA, 25 bp, synthetic construct
  misc_feature 1..25: note = Description of sequence: EGE-YMX-006-A-3'loxP-R2 (Hs3st4-3R)
  source 1..25: mol_type = other DNA
gccatgagtgggttaagctcaagga

SEQ ID NO: 13 - DNA, 25 bp, synthetic construct
  misc_feature 1..25: note = Description of sequence: EGE-YMX-006-A-5'loxP-F2 (Hs3st4-5F)
  source 1..25: mol_type = other DNA
aatcaggggtcagaaaaccgcctta

SEQ ID NO: 14 - DNA, 25 bp, synthetic construct
  misc_feature 1..25: note = Description of sequence: EGE-YMX-006-A-5'loxP-R2 (Hs3st4-5R)
  source 1..25: mol_type = other DNA
ggctgtgggaaaaagcccaaatgat

SEQ ID NO: 15 - DNA, 25 bp, synthetic construct
  misc_feature 1..25: note = Description of sequence: EGE-YMX-006-A-5'loxP-F2 (Hs3st4-5F)
  source 1..25: mol_type = other DNA
aatcaggggtcagaaaaccgcctta

SEQ ID NO: 16 - DNA, 25 bp, synthetic construct
  misc_feature 1..25: note = Description of sequence: EGE-YMX-006-A-3'loxP-R2(Hs3st4-5R)
  source 1..25: mol_type = other DNA
gccatgagtgggttaagctcaagga

SEQ ID NO: 17 - DNA, 27 bp, synthetic construct
  misc_feature 1..27: note = Description of sequence: EGE-ZY3-002-B-5'loxP-F (Hs3st5-5F)
  source 1..27: mol_type = other DNA
gttcctacaatcagcacgatgaaagtg

SEQ ID NO: 18 - DNA, 24 bp, synthetic construct
  misc_feature 1..24: note = Description of sequence: EGE-ZY3-002-B-5'loxP-R (Hs3st5-5R)
  source 1..24: mol_type = other DNA
ggatgctgaagggcaaagaggagg

SEQ ID NO: 19 - DNA, 25 bp, synthetic construct
  misc_feature 1..25: note = Description of sequence: EGE-ZY3-002-B-3'loxP-F (Hs3st5-3F)
  source 1..25: mol_type = other DNA
aagcgatcaaaccacacagcattgg

SEQ ID NO: 20 - DNA, 27 bp, synthetic construct
  misc_feature 1..27: note = Description of sequence: EGE-ZY3-002-B-3'loxP-R (Hs3st5-3R)
  source 1..27: mol_type = other DNA
tgttgctgctctgcctgtgtcattctg

SEQ ID NO: 21 - DNA, 27 bp, synthetic construct
  misc_feature 1..27: note = Description of sequence: EGE-ZY3-002-B-5'loxP-F (Hs3st5-5F)
  source 1..27: mol_type = other DNA
gttcctacaatcagcacgatgaaagtg

SEQ ID NO: 22 - DNA, 24 bp, synthetic construct
  misc_feature 1..24: note = Description of sequence: EGE-ZY3-002-B-5'loxP-R (Hs3st5-5R)
  source 1..24: mol_type = other DNA
ggatgctgaagggcaaagaggagg

SEQ ID NO: 23 - DNA, 27 bp, synthetic construct
  misc_feature 1..27: note = Description of sequence: EGE-ZY3-002-B-5'loxP-F (Hs3st5-5F)
  source 1..27: mol_type = other DNA
gttcctacaatcagcacgatgaaagtg

SEQ ID NO: 24 - DNA, 27 bp, synthetic construct
  misc_feature 1..27: note = Description of sequence: EGE-ZY3-002-B-3'loxP-R (Hs3st5-5R)
  source 1..27: mol_type = other DNA
tgttgctgctctgcctgtgtcattctg

SEQ ID NO: 25 - DNA, 22 bp, synthetic construct
  misc_feature 1..22: note = Description of sequence: B-EM-045-WT-F
  source 1..22: mol_type = other DNA
agtcgctctgagttgttatcag

SEQ ID NO: 26 - DNA, 27 bp, synthetic construct
  misc_feature 1..27: note = Description of sequence: B-EM-045-WT-R
  source 1..27: mol_type = other DNA
tgagcatgtctttaatctacctcgatg

SEQ ID NO: 27 - DNA, 21 bp, synthetic construct
  misc_feature 1..21: note = Description of sequence: B-EM-045-Mut-F
  source 1..21: mol_type = other DNA
ctctatgacctgctgctggag

SEQ ID NO: 28 - DNA, 22 bp, synthetic construct
  misc_feature 1..22: note = Description of sequence: B-EM-045-Mut-R
  source 1..22: mol_type = other DNA
acgggaagcaatagcatgatac

SEQ ID NO: 29 - DNA, 25 bp, synthetic construct
  misc_feature 1..25: note = Description of sequence: EGE-YMX-006-A-5'loxP-F
  source 1..25: mol_type = other DNA
aaatcaggggtcagaaaaccgcctt

SEQ ID NO: 30 - DNA, 25 bp, synthetic construct
  misc_feature 1..25: note = Description of sequence: EGE-YMX-006-A-5'loxP-R
  source 1..25: mol_type = other DNA
tggcaaattctccttgcctcctgtt

(19) United States
(12) Patent Application Publication
(10) Pub. No.: US 2025/0113809 A1
(43) Pub. Date: Apr. 10, 2025

(54) GENETICALLY MODIFIED MOUSE MODELS AND METHODS OF USE THEREOF

(71) Applicant: University of South Florida, Tampa, FL (US)

(72) Inventor: Lianchun Wang, Tampa, FL (US)

(21) Appl. No.: 18/779,510

(22) Filed: Jul. 22, 2024

Related U.S. Application Data
(60) Provisional application No. 63/514,971, filed on Jul. 21, 2023.

(51) Int. Cl. A01K 67/0276; C12N 9/10; C12N 9/22; C12N 15/11; C12N 15/90

(52) U.S. Cl. CPC A01K 67/0276; C12N 9/13; C12N 9/22; C12N 15/11; C12N 15/907; C12Y 208/02023; A01K 2217/075; A01K 2227/105; A01K 2267/0312; C12N 2310/20

(57) ABSTRACT

The present disclosure relates to heparan sulfate transgenic models and methods of use thereof.

### CROSS-REFERENCE TO RELATED APPLICATION

This application claims priority to, and the benefit of, U.S. Provisional Patent Application No. 63/514,971, filed Jul. 21, 2023, entitled “Conditional Hs3st1, Hs3st4, and Hs3st5 mouse lines,” which is incorporated by reference herein in its entirety.

### STATEMENT REGARDING FEDERALLY SPONSORED RESEARCH

This invention was made with Government Support under Grant No. 5U01CA225784 awarded by the National Institutes of Health. The Government has certain right in the invention.

### REFERENCE TO SEQUENCE LISTING

The sequence listing submitted on Jul. 22, 2024, as an .XML file entitled “11001-194US1_ST26” created on Jul. 19, 2024, and having a file size of 27,737 bytes is hereby incorporated by reference pursuant to 37 C.F.R. § 1.52 (e) (5).

### FIELD

The present disclosure relates to heparan sulfate transgenic models and methods of use thereof.

### BACKGROUND

3-O-sulfation is a rare modification of heparan sulfate. Recent studies have shown that different 3-O-sulfation modification enzymes (heparan sulfate O-sulfotransferases, Hs3sts) can generate different 3-O-sulfated heparan sulfate structures that have critical and unique biological functions. Currently, the important biological roles of Hs3sts have not been well investigated due to a lack of suitable testing models. Thus, what is needed is the generation of model systems to investigate and understand the biological roles of Hs3sts. The present disclosure provides essential tools to address the biology and related diseases of 3-O-sulfation of heparan sulfate and related Hs3sts.

### SUMMARY

The present disclosure provides mouse model systems with conditional deletion of heparan sulfate-related genes. The present disclosure also provides methods of generating a mouse model system with a conditional deletion of a heparan sulfate-related gene. The present disclosure also provides methods of screening for therapeutic agents using a mouse model system with a conditional deletion of a heparan sulfate-related gene. The present disclosure also provides methods of treating and/or preventing a heparan-sulfate related disease.

In some aspects, disclosed herein is a transgenic mouse comprising a nucleic acid sequence comprising a heterologous heparan sulfate-glucosamine 3-sulfotransferase (HS3ST) gene and at least two loxP nucleic acid sequences, and wherein a native HS3ST gene is deleted in the transgenic mouse.

In some embodiments, the native HS3ST gene or the heterologous HS3ST gene comprises HS3ST1, HS3ST4, or HS3ST5. In some embodiments, the transgenic mouse further comprises a transgenic gene encoding a Cre recombinase. In some embodiments, the Cre recombinase is an inducible Cre recombinase or conditional Cre recombinase.

In some aspects, disclosed herein is a method of generating a transgenic HS3ST mouse, the method comprising targeting a native HS3ST gene within a mouse with one or more single guide ribonucleic acid (sgRNA) sequences and a CRISPR-Cas9 system, wherein the CRISPR-Cas9 system excises the native HS3ST gene, and inserting a nucleic acid sequence to replace the native HS3ST gene, wherein the nucleic acid sequence comprises a heterologous HS3ST gene and at least two loxP nucleic acid sequences.

In some aspects, disclosed herein is a method of screening for a therapeutic agent for treating or preventing a heparan sulfate-related disease, the method comprising breeding a heparan sulfate-glucosamine 3-sulfotransferase (HS3ST) transgenic mouse with a Cre recombinase transgenic mouse to generate an HS3ST x Cre transgenic mouse, wherein the HS3ST transgenic mouse and the HS3ST x Cre transgenic mouse comprise a nucleic acid sequence comprising a heterologous HS3ST gene and at least two loxP nucleic acid sequences, activating expression of a Cre recombinase within the HS3ST x Cre transgenic mouse, wherein the Cre recombinase excises the heterologous HS3ST gene, inducing one or more phenotypes in the HS3ST x Cre transgenic mouse, administering the therapeutic agent to the HS3ST x Cre transgenic mouse, and detecting an improvement of the one or more phenotypes in the HS3ST x Cre transgenic mouse relative to an untreated transgenic mouse.

In some embodiments, the method of any preceding aspect further comprises breeding the transgenic HS3ST mouse with a Cre recombinase transgenic mouse to generate an HS3ST x Cre transgenic mouse. In some embodiments, the transgenic HS3ST mouse further comprises a nucleic acid sequence encoding a Cre recombinase. In some embodiments, the native HS3ST gene or the heterologous HS3ST gene comprises HS3ST1, HS3ST4, or HS3ST5.

In some embodiments, the method of any preceding aspect excises a native HS3ST1 gene or a native HS3ST4 gene by targeting a nucleic acid sequence upstream of intron 1 and intron 2 of exon 2, and a nucleic acid sequence downstream of the native HS3ST1 gene or the native HS3ST4 gene. In some embodiments, the method of any preceding aspect excises a native HS3ST5 gene by targeting a nucleic acid sequence upstream of intron 5 and intron 6 of exon 6, and a nucleic acid sequence downstream of the native HS3ST5 gene.

In some embodiments, the Cre recombinase is an inducible Cre recombinase or conditional Cre recombinase. In some embodiments, the Cre recombinase excises the heterologous HS3ST gene.

In some embodiments, the HS3ST x Cre mouse comprises a conditional deletion of the heterologous HS3ST gene. In some embodiments, the one or more phenotypes are caused by a conditional deletion of the heterologous HS3ST gene. In some embodiments, the conditional deletion comprises a cell-specific deletion, a tissue-specific deletion, a developmental-specific deletion, a disease-stage specific deletion, or a combination thereof.

### BRIEF DESCRIPTION OF FIGURES

The accompanying figures, which are incorporated in and constitute a part of this specification, illustrate several aspects described below.

FIGS. 1A and 1B show that Hs3st4(+/−) mice exhibited learning deficits in the Morris water maze test. Data were analyzed using an unpaired t-test. Histograms represent the mean±S.E.M (n=10-15 mice per group).

FIGS. 2A and 2B show that Hs3st5(+/−) mice exhibited learning and memory deficits in the Morris water maze test, with a sex-related difference in acquisition memory in 6-month-old mice. Data were analyzed using an unpaired t-test. Histograms represent the mean±S.E.M (n=10-15 mice per group).

FIGS. 3A, 3B, 3C, 3D, 3E, 3F, and 3G show six- and three-month-old male (FIGS. 3A and 3E) and female (FIGS. 3B and 3F) Hs3st4(+/−) mice exhibited hyperactivity and ADHD-like phenotypes in the marble burying test, with no sex-related differences observed (FIGS. 3C and 3G). Red asterisks indicate buried marbles (FIG. 3D). **** p<0.0001, *** p<0.001, * p<0.05. Data were analyzed using an unpaired t-test. Histograms represent mean±S.E.M (n=10-15 mice per group).

FIGS. 4A, 4B, 4C, 4D, 4E, 4F, and 4G show six- and three-month-old male (FIGS. 4A and 4E) and female (FIGS. 4B and 4F) Hs3st4+/− mice exhibited repetitive and ADHD-like phenotypes in the nestlet shredding test, with no sex-related difference (FIGS. 4C and 4G). Unshredded nestlet weight was measured after 1 hour (FIG. 4D). * p<0.05. Data were analyzed using an unpaired t-test. Histograms represent mean±S.E.M (n=10-15 mice per group).

FIGS. 5A, 5B, 5C, 5D, 5E, and 5F show six- and three-month-old male (FIGS. 5A and 5D) and female (FIGS. 5B and 5E) Hs3st5+/− mice exhibited hyperactivity in the marble burying test, without sex-related differences (FIGS. 5C and 5F). **** p<0.0001, *** p<0.001, ** p<0.01. Data were analyzed using an unpaired t-test. Histograms represent mean±S.E.M (n=10-15 mice per group).

FIGS. 6A, 6B, 6C, 6D, 6E, and 6F show six- and three-month-old male (FIGS. 6A and 6D) and female (FIGS. 6B and 6E) Hs3st5+/− mice exhibited repetitive and compulsive-like behaviors in the nestlet shredding test, without sex-related differences (FIGS. 6C and 6F). *** p<0.001, ** p<0.01. Data were analyzed using an unpaired t-test. Histograms represent mean±S.E.M (n=10-15 mice per group).

FIGS. 7A, 7B, 7C, 7D, and 7E show that microglia exhibit alterations in numbers, morphology, and function in the prefrontal cortex of male Hs3st4(+/−) mice. (scale bar: 50 μm). White arrowheads indicate amoeboid microglia. Histograms represent mean±S.E.M. *p<0.05, ** p<0.01, and *** p<0.001.

FIGS. 8A, 8B, 8C, 8D, 8E, and 8F show that Hs3st4 deficiency affects the structural integrity of cerebral vasculature. Co-immunostaining of Podocalyxin and Claudin in the prefrontal cortex of WT and Hs3st4(+/−) male mice. (scale bar: 50 μm). Histograms represent mean±S.E.M. (n=5 per group), **** p<0.0001.

FIG. 9 shows the GFAP immunostaining of the prefrontal cortex in 6-month-old WT and Hs3st4(+/−) male mice. Data were analyzed using an unpaired t-test. Histogram represents mean±S.E.M (n=3 mice per group.

FIGS. 10A, 10B, 10C, 10D, 10E, 10F, and 10G show that synaptogenesis is affected in Hs3st4(+/−) male mice. Immunostaining of Synaptophysin, PSD95, and MAP2 in the prefrontal cortex of WT and Hs3st4(+/−) male mice. (scale bar: 50 μm). Histograms represent mean±S.E.M. (n=5). * p<0.05, ** p<0.01, *** p<0.001.

FIG. 11 shows the coimmunostaining of NeuN and TH in the prefrontal cortex of male WT and Hs3st4(+/−) mice. (scale bar: 50 μm). Histograms represent mean±S.E.M. (n=4). *** p<0.001.

FIG. 12 shows the β-tubulin III immunostaining in the dentate gyrus of WT and Hs3st5(+/−) male mice. (scale bar: 50 μm). Arrowheads indicate normal neurons, and arrows denote apolar neurons in the dentate gyrus.

FIG. 13 shows the co-immunostaining of MAPII, synaptophysin, and PSD95 in the cortex of WT and Hs3st5(+/−) male mice. (scale bar: 50 μm). Heterogeneity in the morphology and distribution of neurons and synapses is observed in the cortex of Hs3st5(+/−) mice.

FIG. 14 shows the knockout of Hs3st1 and Ext1 attenuated neuronal uptake of tau-488 fibrils. Mice were injected with 500 ng of FL tau-488 fibrils (green). Two days after the injection, brains were examined by staining brain sections with anti-MAPII antibody to label neurons (red) and counterstained with DAPI (blue). Arrowheads designate FL tau fibrils associated with neurons. More and larger tau-488 fibril aggregates were seen in the wildtype neurons, significantly reduced in number and size in Hs3st1-knockout neurons, and barely seen in the Ext1-knockout neuron. Four mice per group, and 4 images of each mouse in total of 16 data points per group were presented. Unpaired t-test for the two-group comparison. Hs3st1^{iKO} are the tamoxifen-treated mice in which the expression of Hs3st1 is globally knockout. Ext1^{nKO} are the neuron-specific Ext1 knockout mice in which neuronal HS expression is diminished and were included as the background control of HS-dependent tau uptake 1.

FIGS. 15A, 15B, 15C, 15D, and 15E show the generation of the conditional Hs3st1 mice. FIG. 15B shows the gel image of the PCR products using the primer set EGE-YQH-075-B-5′loxP-F/EGE-YQH-075-B-5′loxP-R. FIG. 15C shows the gel image of the PCR product using EGE-YQH-075-B-3′loxP-F/EGE-YQH-075-B-3′loxP-R. FIG. 15D shows the gel image of the PCR product using the primer set B-EM-045-Mut-F/B-EM-045-Mut-R. FIG. 15E shows the gel image of the PCR product using the primer set B-EM-045-WT-F/B-EM-045-WT-R. DNA is separated by gel electrophoresis on a 2% agarose gel. WT: wild-type control; H₂O: blank control.

FIG. 16 shows a schematic of the Cre-mediated conditional Hs3st1 deletion.

FIG. 17 shows the generation of conditional Hs3st4 mice.

FIG. 18 shows a schematic of the Cre-mediated conditional Hs3st4 deletion.

FIG. 19 shows the generation of conditional Hs3st5 mice.

FIG. 20 shows a schematic of the Cre-mediated conditional Hs3st5 deletion.

FIGS. 21A, 21B, 21C, and 21D show the generation of the conditional Hs3st4 mice.

FIG. 21A shows the gel image of the PCR products using primer set EGE-YMX-006-A-5′loxP-F/EGE-YMX-006-A-5′loxP-R. FIG. 21B shows the gel image of the PCR products using primer set EGE-YMX-006-A-3′loxP-F2/EGE-YMX-006-A-3′loxP-R2. FIG. 21C shows the gel image of the PCR products using primer set B-EM-045-Mut-F/B-EM-045-Mut-R. FIG. 21D shows the gel image of the PCR products using primer set B-EM-045-WT-F/B-EM-045-WT-R.

FIGS. 22A, 22B, 22C, 22D, 22E, and 22F show the generation of the conditional Hs3st5 mice. FIGS. 22A and 22B show the gel image of the PCR products using primer set EGE-ZY3-002-B-3′loxP-F/EGE-ZY3-002-B-3′loxP-R. FIGS. 22C and 22D show the gel image of the PCR products using primer set EGE-ZY3-002-B-5′loxP-F EGE-ZY3-002-B-5′loxP-R. FIG. 22E shows the gel image of the PCR products using primer set B-EM-045-Mut-F/B-EM-045-Mut-R. FIG. 22F shows the gel image of the PCR products using primer set B-EM-045-WT-F/B-EM-045-WT-R.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction below, above, or in between the given ranges as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of a heparan sulfate-related disease), during early onset (e.g., upon initial signs and symptoms of a heparan sulfate-related disease), or after an established development of a heparan sulfate-related disease.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective amount” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

As used herein, the term “polymerase chain reaction” (“PCR”) refers to a method for increasing the concentration of a segment of a target sequence in a mixture of genomic DNA without cloning or purification. This process for amplifying the target sequence typically consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times to obtain a high concentration of an amplified segment of the desired target sequence. Unless otherwise noted, PCR, as used herein, also includes variants of PCR such as allele-specific PCR, asymmetric PCR, hot-start PCR, ligation-mediated PCR, multi-plex-PCR, reverse transcription PCR, or any of the other PCR variants known to those skilled in the art.

A “promoter,” as used herein, refers to a sequence in DNA that mediates the initiation of transcription by an RNA polymerase. Transcriptional promoters may comprise one or more of a number of different sequence elements as follows: 1) sequence elements present at the site of transcription initiation; 2) sequence elements present upstream of the transcription initiation site and; 3) sequence elements downstream of the transcription initiation site. The individual sequence elements function as sites on the DNA, where RNA polymerases and transcription factors facilitate positioning of RNA polymerases on the DNA bind.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

As used herein, the term, “deletion,” also called gene deletion, deficiency, or deletion mutation, refers to part of a chromosome or a sequence of DNA being left out during DNA replication. Deletion, or gene deletions can cause any number of nucleotides to be deleted from a single base to an entire piece of chromosome. Variants comprising deletions relative to a nucleotide sequence are contemplated herein. A “deletion” refers to a change in the nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated.

A “transgene” refers to a non-native gene or an artificial gene, manipulated by molecular biology techniques, that is incorporated into a vector (such as, for example a viral vector or plasmid) along with all the appropriate elements critical from gene expression. Generally, the transgene is originally derived from a different species relative to the vector species or a host species.

The term “screening” refers to a method associated with drug discovery in which data processing/control software, liquid handling devices, and sensitive detectors can allow for quick conductions of chemical, genetic, or pharmacological tests. This process allows one to quickly recognize active compounds, antibodies, or genes that modulate a particular biomolecular pathway. The results of these processes provide starting points for drug design.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (Sec, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

The term “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein may be entirely synthetic. In another example, a target polynucleotide for cleavage by a Cas endonuclease may be of a different organism than that of the Cas endonuclease. In another example, a Cas endonuclease and guide RNA may be introduced to a target polynucleotide with an additional polynucleotide that acts as a template or donor for insertion into the target polynucleotide, wherein the additional polynucleotide is heterologous to the target polynucleotide and/or the Cas endonuclease.

As used herein, a “mutation” refers to changing the structure of a gene, resulting in a variant form that may be transmitted to later generations. A mutation is caused by the alteration of single nucleotides in DNA, or the deletion, insertion, or rearrangement of larger sections of genes. A mutation can lead to the expression of a protein that has been changed physically or functionally leading to lethality, non-lethal dysfunction effects, or no effects.

“Downstream” refers to a direction of transcription, the direction of transcription being from a promoter sequence to a RNA-encoding sequence. For a template strand of a double-stranded DNA molecule, the direction of transcription is 3′ to 5′. For a nontemplate strand of the double-stranded DNA molecule, the direction of transcription is 5′ to 3′.

“Upstream” refers to a direction opposite the direction of transcription. “Upstream” and “downstream” may be used in reference to either strand of a double-stranded DNA molecule even when relative to a sequence on one strand of a double-stranded DNA molecule.

As used herein, “wild-type” refers to the genetic and physical characteristics of the typical form of a species as it occurs in nature. A wild-type or wild type characteristic is conceptualized as a product of the standard “normal” allele at a gene locus, in contrast to that produced by a non-standard “mutant” allele.

### Heparan Sulfate-Glucosamine 3-Sulfotransferase (HS3ST) Transgenic Mice

The present disclosure provides transgenic model systems with conditional deletion of heparan sulfate-related genes.

“Transgenic model systems” refer to biological models that have had DNA from a different or foreign source integrated into their genomic DNA. The foreign DNA is put into the nucleus of a cell, most commonly a fertilized oocyte of a chosen animal. The foreign DNA then becomes part of every cell or tissue of the model system. These biological models can be used in a laboratory setting to study and/or understand biological mechanisms and diseases. In some embodiments, the transgenic model system herein comprises a non-human transgenic animal including, but not limited to a primate, a canine, a feline, or a rodent. In some embodiments, the transgenic model system herein comprises a rodent including, but not limited to a mouse or a rat.

In some aspects, disclosed herein is a transgenic mouse comprising a nucleic acid sequence comprising a heterologous heparan sulfate-glucosamine 3-sulfotransferase (HS3ST) gene and at least two loxP nucleic acid sequences, and wherein a native HS3ST gene is deleted in the transgenic mouse. In some embodiments, the native HS3ST gene or the heterologous HS3ST gene comprises HS3ST1, HS3ST4, or HS3ST5. In some embodiments, the nucleic acid sequence encodes the heterologous HS3ST operably linked to a promoter. In some embodiments, the promoter includes a tissue-specific promoter (such as, for example liver-specific promoters, cardiac-specific promoters, muscle-specific promoters, brain-specific promoters, cancer-specific promoters, germ-line-specific promoters, and immune cell-specific promoters), an inducible promoter (such as, for example positive inducible promoters, negative inducible promoters, chemically inducible promoters, temperature inducible promoters, and light inducible promoters), or a constitutive promoter, which remain active in a cell under all conditions. As used herein, “operably linked” refers to nucleic acids being placed in a functional relationship with another nucleic acid sequence. “Operably linked” also refers to DNA sequences being linked in a contiguous manner.

In some embodiments, the transgenic mouse further comprises a transgene encoding a Cre recombinase. A Cre recombinase is a bacteria-derived enzyme that recognizes a portion of a nucleic acid sequence called loxP sites, and deletes the nucleotides between two loxP sites. It should be appreciated that there are several different types of Cre recombinases and loxP variant sequences known in the art that can be used and interchanged to create more sophisticated recombination events. In some embodiments, any Cre recombinase and loxP sequence known in the art can be integrated and/or expressed in the transgenic model system disclosed herein. In some embodiments, the Cre recombinase is an inducible Cre recombinase or a conditional Cre recombinase. In some embodiments, the Cre recombinase may be introduced into the organs of chosen model system, including but not limited to liver, lungs, kidney, heart, pancreas, skeletal muscles, brain, and digestive organs.

### Methods of Generating an HS3ST Transgenic Mouse

The present disclosure also provides methods of generating a mouse model system with a conditional deletion of a heparan sulfate-related gene.

In some aspects, disclosed herein is a method of generating a transgenic HS3ST mouse, the method comprising targeting a native HS3ST gene within a mouse with one or more single guide ribonucleic acid (sgRNA) sequences and a CRISPR-Cas9 system, wherein the CRISPR-Cas9 system excises the native HS3ST gene, and inserting a nucleic acid sequence to replace the native HS3ST gene, wherein the nucleic acid sequence comprises a heterologous HS3ST gene and at least two loxP nucleic acid sequences.

“CRISPR” (Clustered Regularly Interspaced Short Palindromic Repeats) loci refers to certain genetic loci encoding components of DNA cleavage systems, for example, used by bacterial and archacal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

A Cas protein includes proteins encoded by a gene in a cas locus and includes adaptation molecules as well as interference molecules. An interference molecule of a bacterial adaptive immunity complex includes endonucleases. A Cas endonuclease described herein comprises one or more nuclease domains. A Cas endonuclease includes but is not limited to: the novel Cas-alpha protein disclosed herein, a Cas9 protein, a Cas12a (Cpf1) protein, a Cas12b (C2c1) protein, a Cas13a (C2c2) protein, a Cas12c (C2c3) protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. The Cas-alpha endonucleases of the disclosure include those having one or more RuvC nuclease domains. A Cas protein is further defined as a functional fragment or functional variant of a native Cas protein, or a protein that shares at least 30%, between 30% and 35%, at least 35%, between 35% and 40%, at least 40%, between 40% and 45%, at least 45%, between 45% and 50%, at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity of the native sequence.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site (such as, for example the HS3ST gene of any preceding aspect), enabling the Cas endonuclease to recognize, optionally bind to, and optionally nick or cleave (introduce a single or double-strand break) the DNA target site. In some embodiments, the CRISPR-Cas9 system excises the native HS3ST gene and inserts the heterologous HS3ST gene in the mouse genome.

It should be understood that the transgenic HS3ST mouse comprises the heterologous HS3ST gene with a loxP site located at the 5′ end and 3′ end, which is also referred to “floxed HS3ST”, “HS3ST flox/flox”, or “HS3ST fl/fl”. In some embodiments, the method of any preceding aspect further comprises breeding the transgenic HS3ST mouse with a Cre recombinase transgenic mouse to generate an HS3ST x Cre transgenic mouse. The transgenic HS3ST mouse of any preceding aspect can be bred with any characterized Cre recombinase mouse line known in the art. Examples of characterized Cre recombinase mouse lines include those disclosed by the Jackson Laboratory (See The Jackson Laboratory Characterized Cre Lines, https://www.jax.org/research-and-faculty/resources/cre-repository/characterized-cre-lines-jax-cre-resource). In some embodiments, the transgenic HS3ST mouse further comprises a nucleic acid sequence encoding a Cre recombinase. In some embodiments, the Cre recombinase is an inducible Cre recombinase or conditional Cre recombinase. The Cre recombinase of any preceding aspect targets and cuts at the loxP sites to excise the HS3ST gene. In some embodiments, the Cre recombinase excises the heterologous HS3ST gene. In some embodiments, the native HS3ST gene or the heterologous HS3ST gene comprises HS3ST1, HS3ST4, or HS3ST5.

The present disclosure provides an experimental design to conditionally knockout the exon 2 (for Hs3st1 and Hs3st4) or exon 6 (for Hs4st5) by the Cre-loxP system. The intron 1-2 for exon 2, or 5-6 for exon 6, and downstream of the targeted exons are large, so the insertion of loxP elements is expected not to interfere with mRNA splicing. To minimize the possibility of disruption of Hs3st expression, both loxP sites were inserted into non-conserved regions. In some embodiments, the method of any preceding aspect excises a native HS3ST1 gene or a native HS3ST4 gene by targeting a nucleic acid sequence upstream of intron 1 and intron 2 of exon 2, and a nucleic acid sequence downstream of the native HS3ST1 gene or the native HS3ST4 gene. In some embodiments, the method of any preceding aspect excises a native HS3ST5 gene by targeting a nucleic acid sequence upstream of intron 5 and intron 6 of exon 6, and a nucleic acid sequence downstream of the native HS3ST5 gene. As used herein, an “intron” refers to a region that resides within a gene but does not remain in the final mature messenger RNA (mRNA) molecule following transcription of said gene and does not code for amino acids that make up the protein encoded by said gene. As used herein, an “exon” refers to coding section of an mRNA transcript, or the DNA encoding said mRNA, that is translated into protein. Exons are generally separated by intron sequences and are joined together prior to transcription during the process termed “splicing”.

In some embodiments, the HS3ST x Cre mouse comprises a conditional deletion of the heterologous HS3ST gene. Conditional gene deletion or knockout is a technique used to eliminate a specific gene, such as, for example a heparan sulfate gene (including, but not limited to HS3ST1, HS3ST4, or HS3ST5, in a specific organ, tissue, or cell at a specific time. It should be noted that conditional gene deletion differs from traditional gene deletion techniques because it targets specific genes at specific times rather than deletion occurring from the beginning of life. It should also be noted that use of conditional gene deletion strategies eliminates many side effects caused by traditional gene deletion. For example, traditional gene deletion can cause embryonic death when said gene is deleted early in life. However, using a conditional deletion strategy that deletes said gene later in life, for instance during adolescence or adulthood, allows for those with skills in the art to study and understand roles and diseases related to said gene.

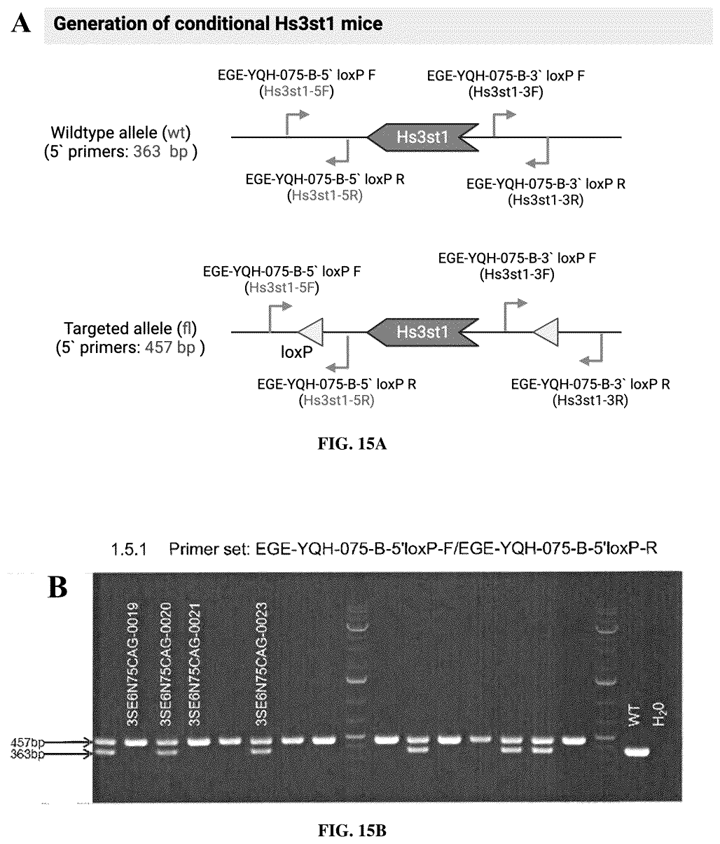

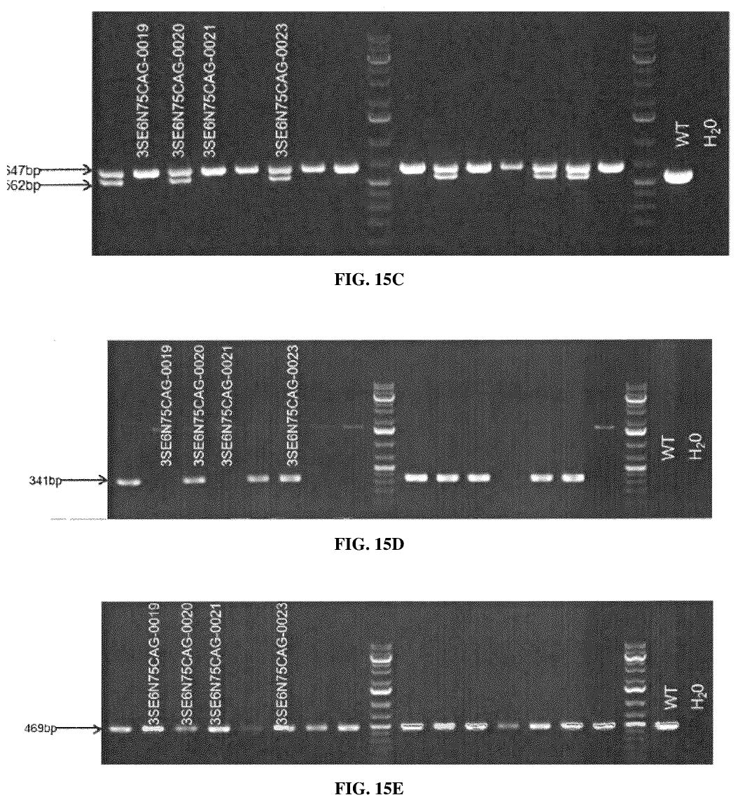

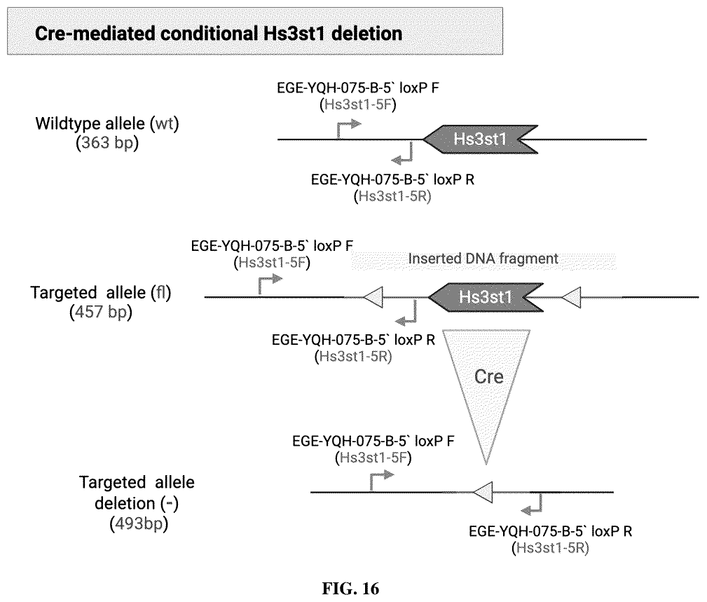

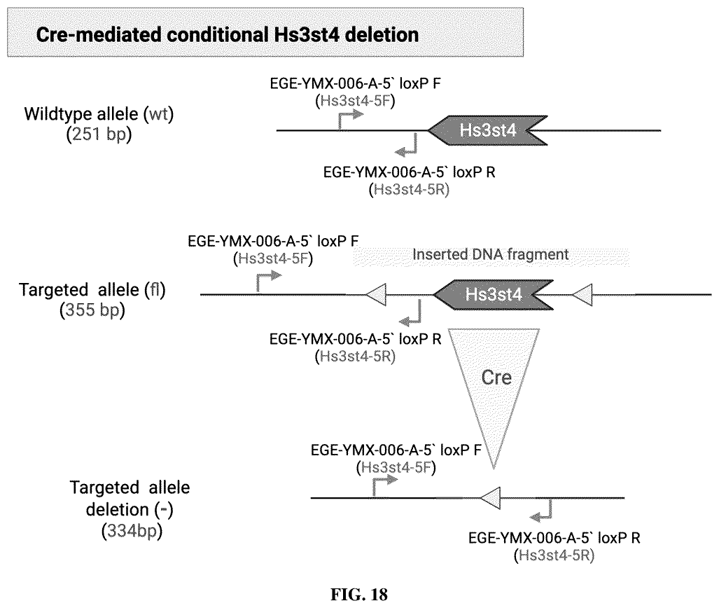

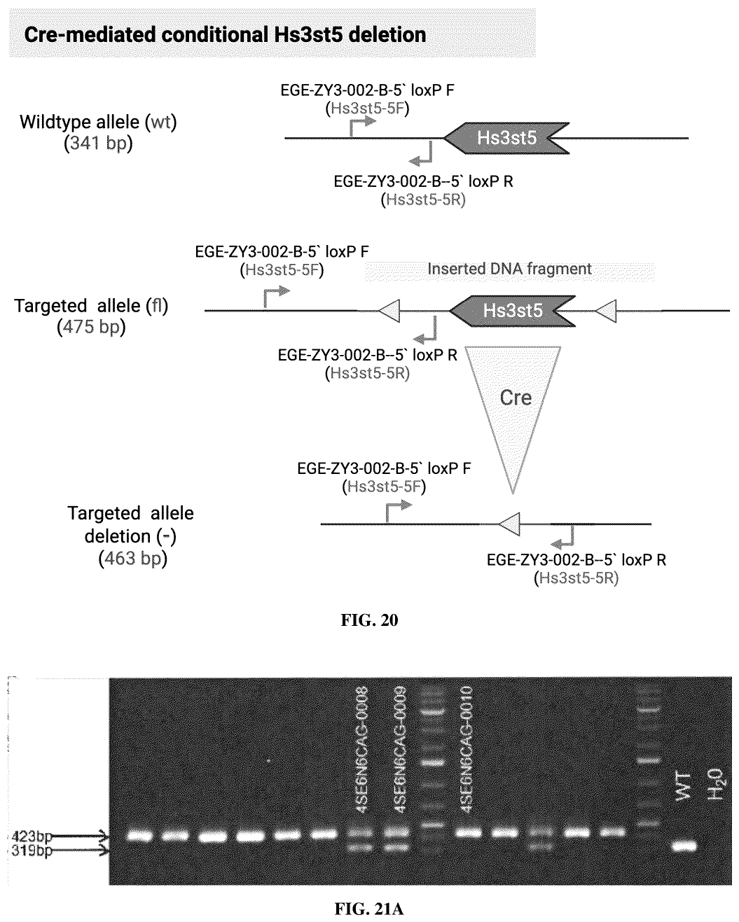

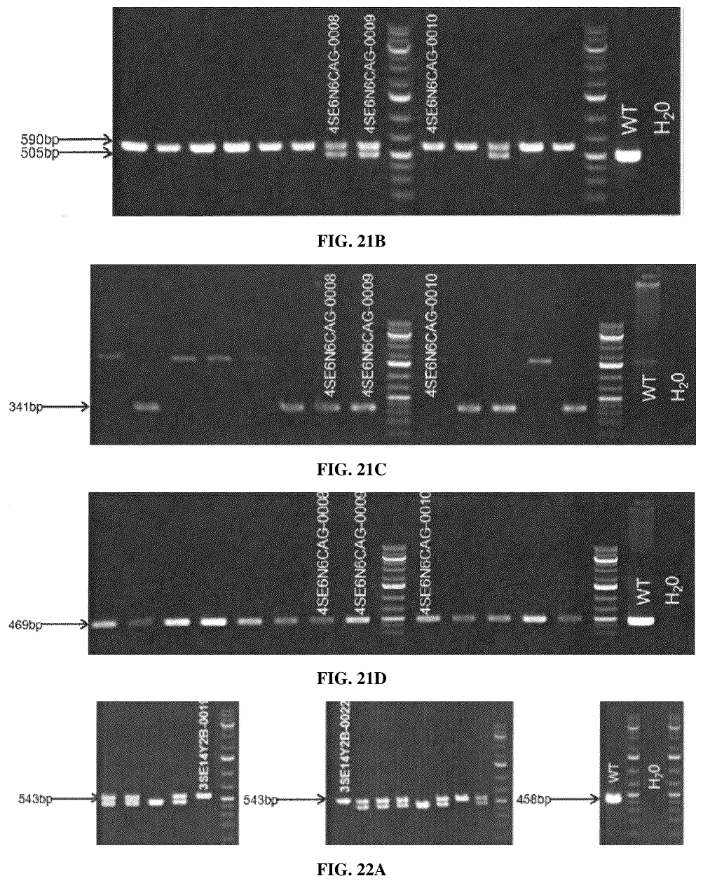

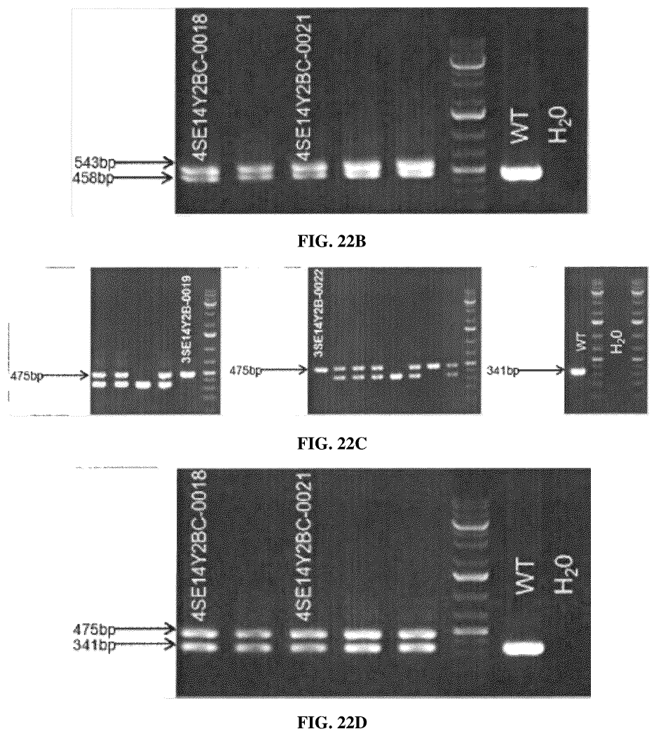

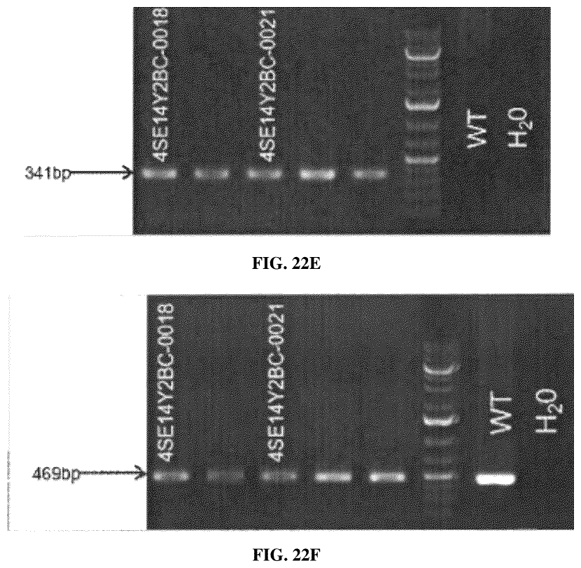

Generating a transgenic model requires confirmation that the animal model has been genetically modified successfully. Such techniques to confirm genetic modification include, but are not limited to polymerase chain reactions (PCR), western blot, agarose gel electrophoresis, and other molecular techniques. The present disclosure uses PCR and gel electrophoresis techniques to confirm successful generation of HS3ST transgenic mice. Non-limiting examples and protocols of these techniques are demonstrated in Tables 12, 13, 14, 15, 16, 17, and 18; and in FIGS. 15A, 15B, 15C, 15D, 15E, 16, 17, 18, 19, 20, 21A, 21B, 21C, 21D, 22A, 22B, 22C, 22D, 22E, and 22F. The present disclosure also provides the primers used to perform PCRs and confirm HS3ST genotypes. In some embodiments, the method of any disclosed aspect comprises any combination of primer sequences selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or any variants thereof.

In some embodiments, the one or more phenotypes are caused by a conditional deletion of the heterologous HS3ST gene. In some embodiments, the one or more phenotypes include, but are limited to decreased uptake of Tau protein aggregates, and Attention deficit/Hyperactivity Disorder (ADHD)-related symptoms. In some embodiments, the conditional deletion comprises a cell-specific deletion, a tissue-specific deletion, a developmental-specific deletion, a disease-stage specific deletion, or a combination thereof.

### Methods of Screening for a Therapeutic Agent

The present disclosure also provides methods of screening for therapeutic agents using a mouse model system with a conditional deletion of a heparan sulfate-related gene.

In some aspects, disclosed herein is a method of screening for a therapeutic agent for treating or preventing a heparan sulfate-related disease, the method comprising breeding a heparan sulfate-glucosamine 3-sulfotransferase (HS3ST) transgenic mouse with a Cre recombinase transgenic mouse to generate an HS3ST x Cre transgenic mouse, wherein the HS3ST transgenic mouse and the HS3ST x Cre transgenic mouse comprise a nucleic acid sequence comprising a heterologous HS3ST gene and at least two loxP nucleic acid sequences, activating expression of a Cre recombinase within the HS3ST x Cre transgenic mouse, wherein the Cre recombinase excises the heterologous HS3ST gene, inducing one or more phenotypes in the HS3ST x Cre transgenic mouse, administering the therapeutic agent to the HS3ST x Cre transgenic mouse, and detecting an improvement of the one or more phenotypes in the HS3ST x Cre transgenic mouse relative to an untreated transgenic mouse.

In some embodiments, the method comprises generating a transgenic HS3ST mouse of any preceding aspect. In some embodiments, the method of any preceding aspect further comprises breeding the transgenic HS3ST mouse with a Cre recombinase transgenic mouse to generate an HS3ST x Cre transgenic mouse. In some embodiments, the transgenic HS3ST mouse further comprises a nucleic acid sequence encoding a Cre recombinase. In some embodiments, the native HS3ST gene or the heterologous HS3ST gene comprises HS3ST1, HS3ST4, or HS3ST5.

In some embodiments, the method of any preceding aspect excises a native HS3ST1 gene or a native HS3ST4 gene by targeting a nucleic acid sequence upstream of intron 1 and intron 2 of exon 2, and a nucleic acid sequence downstream of the native HS3ST1 gene or the native HS3ST4 gene. In some embodiments, the method of any preceding aspect excises a native HS3ST5 gene by targeting a nucleic acid sequence upstream of intron 5 and intron 6 of exon 6, and a nucleic acid sequence downstream of the native HS3ST5 gene.

In some embodiments, Cre expression is activated using a change in temperature, administering a chemical, such as tamoxifen or doxycycline, to the mouse, or activated at a specific time during life. In some embodiments, the Cre recombinase is an inducible Cre recombinase or conditional Cre recombinase. In some embodiments, the Cre recombinase excises the heterologous HS3ST gene.

In some embodiments, the HS3ST x Cre mouse comprises a conditional deletion of the heterologous HS3ST gene. In some embodiments, the one or more phenotypes are caused by a conditional deletion of the heterologous HS3ST gene. In some embodiments, the method of screening comprises the one or more phenotypes in the HS3ST x Cre transgenic mouse including, but are limited to decreased uptake of Tau protein aggregates, and Attention deficit/Hyperactivity Disorder (ADHD)-related symptoms. In some embodiments, the conditional deletion comprises a cell-specific deletion, a tissue-specific deletion, a developmental-specific deletion, a disease-stage specific deletion, or a combination thereof.

In some embodiments, the therapeutic agent includes, but is not limited to an antibody, peptide, hormone, antibiotic, antiviral, an immunomodulatory agent, an anti-cancer agent, a cell (such as, for example a T cell, a natural killer cell, or a combination thereof), or any biologically active compound or composition thereof. In some embodiments, the method of screening comprises detecting an improvement of the one or more phenotypes in the HS3ST x Cre transgenic mouse relative to an untreated transgenic mouse. As disclosed herein, an improvement comprises an increase in one or more physiological function normally observed in healthy individuals. Said increase can be 1, 2, 3, 4, 5, 6, 7, 8, 9, 10, 15, 20, 25, 30, 35, 40, 45, 50, 55, 60, 65, 70, 75, 80, 85, 90, 95, 100% or more increase in one or more physiological function. An improvement can also comprise a decrease in one or more pathological symptom. Said decrease can be 1, 2, 3, 4, 5, 6, 7, 8, 9, 10, 15, 20, 25, 30, 35, 40, 45, 50, 55, 60, 65, 70, 75, 80, 85, 90, 95, 100% or more decreased in one or more pathological symptom.

In some embodiments, the method of any preceding aspect allows for the treatment and/or prevention of a heparan sulfate-related disease, including but not limited to Alzheimer's disease, Parkinson's disease, prior diseases, ADHD, adult dementia, and childhood dementia.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Hs3st1 Targeting Strategy

After scanning the Hs3st1 gene structure and the size of exons, Exon 2 (ENSMUSE00000375807) was conditionally removed. The deletion of Exon 2 resulted in a null protein.

This design is to conditionally knockout the exon 2 by Cre-loxP system. The intron 1-2 and downstream of Exon 2 are large, so the insertion of loxP elements will not interfere with mRNA splicing. To minimize the possibility of disruption of Hs3st1 expression. Both of the loxP sites are inserted into non-conserved regions.

### Example 2: Hs3st4 Targeting Strategy

After scanning the Hs3st4 gene structure and the size of exons, Exon 2 (ENSMUSE00000669921) was conditionally removed. After Cre-loxP recombination, the removal of Exon 2 (coding 239 aa-449 aa) and 3′UTR leads to exonuclease-mediated degradation of the mRNA, which results in a null protein.

This design is to conditionally knockout the Exon 2 by Cre-loxP. The intron 1-2 and downstream of Exon 2 are large, so the insertion of loxP elements do not interfere with mRNA splicing. To minimize the possibility of disruption of Hs3st4 expression, both of the loxP sites are inserted into non-conserved regions.

### Example 3: Hs3st5 Targeting Strategy

After scanning the Hs3st5 gene structure and size of exons, Exon 6 (ENSMUSE00000357871) was conditionally removed. After Cre-loxP recombination, the removal of Exon 6 deleted the majority of the coding region and 3′UTR. Thus, the absence of 3′UTR led to exonuclease-mediated degradation of the mRNA, which results in a null protein.

This design is to conditionally knockout the exon 6 by Cre-loxP system. Introns 5-6 and downstream of Exon 6 is large, so the insertion of loxP elements does not interfere with mRNA splicing. To minimize the possibility of disruption of Hs3st5 expression, both of the loxP sites are inserted into non-conserved regions.

### Example 4: Deficiency of Hs3st1 Attenuates Heparan Sulfate to Bind Alzheimer's Disease-Important Molecule ApoE

Apolipoprotein E Recognizes Alzheimer's Disease Associated 3-O Sulfation of Heparan Sulfate. Apolipoprotein E (ApoE)'s ε4 alle is the most important genetic risk factor for late onset Alzheimer's Disease (AD). Cell-surface heparan sulfate (HS) is a cofactor for ApoE/LRP1 interaction and the prion-like spread of tau pathology between cells. 3-O-sulfo (3-O—S) modification of HS has been linked to AD through its interaction with tau, and enhanced levels of 3-O-sulfated HS and 3-O-sulfotransferases in the AD brain. The present disclosure characterizes ApoE/HS interactions in wildtype ApoE3, AD-linked ApoE4, and AD-protective ApoE2 and ApoE3-Christchurch. Glycan microarray and SPR assays revealed that all ApoE isoforms recognized 3-O—S. NMR titration localized ApoE/3-O—S binding to the vicinity of the canonical HS binding motif. In cells, the knockout of HS3ST1—a major 3-O sulfotransferase—reduced cell surface binding and uptake of ApoE. 3-O—S is thus recognized by both tau and ApoE, showing that the interplay between 3-O-sulfated HS, tau and ApoE isoforms may modulate AD risk. Angew Chem Int Ed Engl. 2023 Jun. 5; 62 (23): c202212636. PMCID: PMC10430763. PMID: 37014788.

### Example 5: Deficiency of Hs3st1 Attenuates Heparan Sulfate to Bind Alzheimer's Disease-Associated Molecule Tau

3-O-Sulfation of Heparan Sulfate Enhances Tau Interaction and Cellular Uptake. Prion-like transcellular spreading of tau in Alzheimer's Disease (AD) is mediated by tau binding to cell surface heparan sulfate (HS). However, the structural determinants for tau-HS interaction are not well understood. Microarray and SPR assays of structurally defined HS oligosaccharides show that a rare 3-O-sulfation (3-O—S) of HS significantly enhances tau binding. In Hs3st1^{-/-} (HS 3-O-sulfotransferase-1 knockout) cells, reduced 3-O—S levels of HS diminished both cell surface binding and internalization of tau. In a cell culture, the addition of a 3-O—S HS 12-mer reduced both tau cell surface binding and cellular uptake. NMR titrations mapped 3-O—S binding sites to the microtubule binding repeat 2 (R2) and proline-rich region 2 (PRR2) of tau. Tau is only the seventh protein currently known to recognize HS 3-O-sulfation. Our work demonstrates that this rare 3-O-sulfation enhances tau-HS binding and likely the transcellular spread of tau, providing a novel target for disease-modifying treatment of AD and other tauopathies. Zhao J, Zhu Y, Song X, Xiao Y, Su G, Liu X, Wang Z, Xu Y, Liu J, Eliezer D, Ramlall T F, Lippens G, Gibson J, Zhang F, Linhardt R J, Wang L, Wang C. Angew Chem Int Ed Engl. 2020 Jan. 27; 59(5):1818-1827. doi: 10.1002/anic.201913029. Epub 2019 Dec. 10. PMID: 31692167.

### Example 6: Hs3st1 Expression is Elevated in Alzheimer's Disease and is a Genetic Risk Gene for Alzheimer's Disease

Increased 3-O-sulfated heparan sulfate in Alzheimer's disease brain is associated with the genetic risk gene HS3ST1. HS3ST1 is a genetic risk gene associated with Alzheimer's disease (AD) and overexpressed in patients, but how it contributes to the disease progression is unknown. We report the analysis of brain heparan sulfate (HS) from AD and other tauopathies using a LC-MS/MS method. A specific 3-O-sulfated HS displayed sevenfold increase in the AD group (n=14, P<0.0005). Analysis of the HS modified by recombinant sulfotransferases and HS from genetic knockout mice revealed that the specific 3-O-sulfated HS is made by 3-O-sulfotransferase isoform 1 (3-OST-1), which is encoded by the HS3ST1 gene. A synthetic tetradecasaccharide (14-mer) carrying the specific 3-O-sulfated domain displayed stronger inhibition for tau internalization than a 14-mer without the domain, suggesting that the 3-O-sulfated HS is used in tau cellular uptake. Our findings suggest that the overexpression of HS3ST1 gene may enhance the spread of tau pathology, uncovering a previously unidentified therapeutic target for AD. Wang Z, Patel V N, Song X, Xu Y, Kaminski A M, Doan V U, Su G, Liao Y, Mah D, Zhang F, Pagadala V, Wang C, Pedersen L C, Wang L, Hoffman M P, Gearing M, Liu J. Sci Adv. 2023 May 26; 9(21):eadf6232. doi: 10.1126/sciadv.adf6232. Epub 2023 May 26. PMID: 37235665.

### Example 7: Results in Hs3st1/4/5 Mouse Models

Animal Cohort: The conditional knockout mice for Hs3st1/4/5 on a C57BL/6 genetic background were generated through a commercial contract with Biocytogen and belong the laboratory at the University of South Florida (USF). To induce the targeted global gene deletions, these mice were bred with B-CAG-iCreERT2 mice (from Biocytogen) and administered intraperitoneal injections of Tamoxifen (0.2 mg/g body weight). Preliminary observations indicated that the Hs3st series knockout mice exhibited altered activity and anxiety levels in the cage. These mice were further characterized by assessing their physiological and histo-anatomical phenotypes. Furthermore, the mice underwent additional analysis using various models to evaluate hyperactivity, anxiety, social communication, and cognition. Histological studies were then conducted to identify any neuropathological changes.

Behavioral Studies: All behavioral tests were conducted with male and female knockout (KO) and wild-type mice at 3 and 6 months of age (n=10-15 per group).

### Spatial Learning and Memory

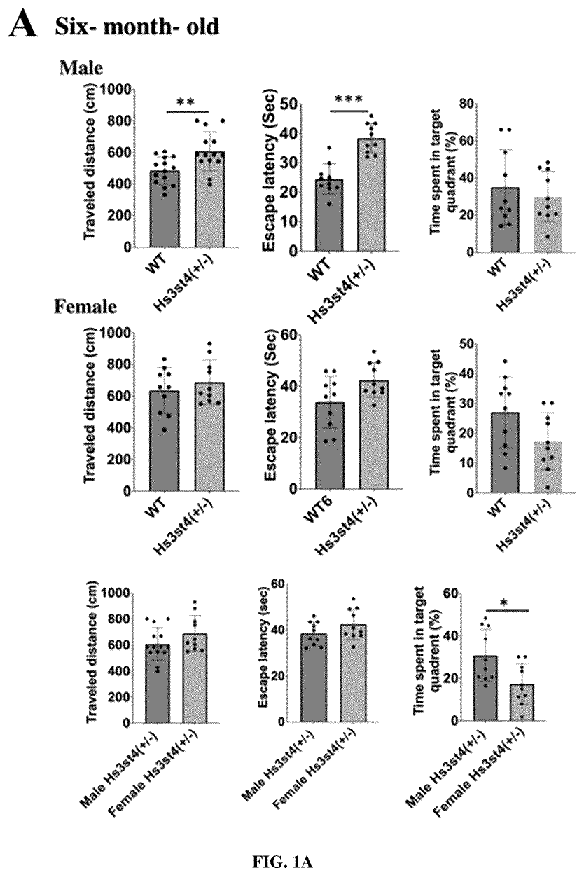

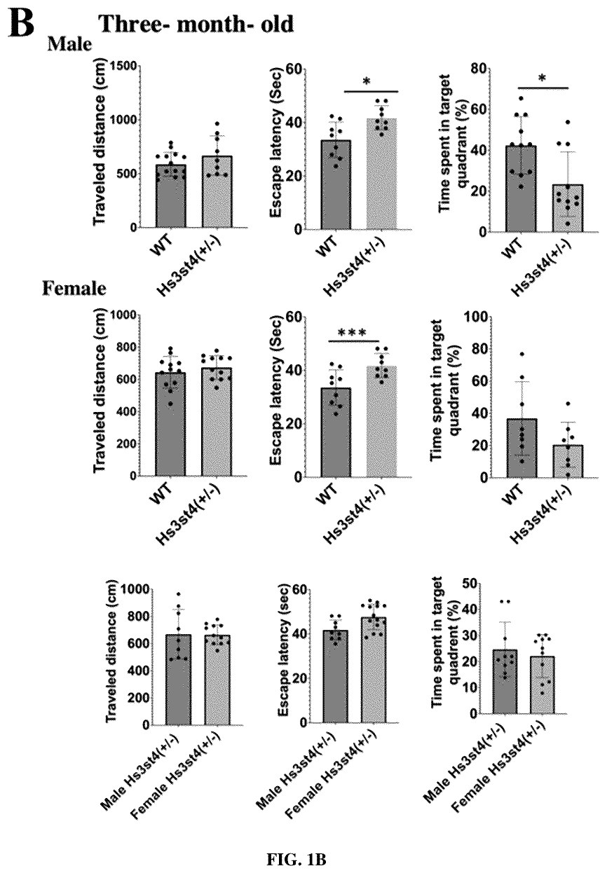

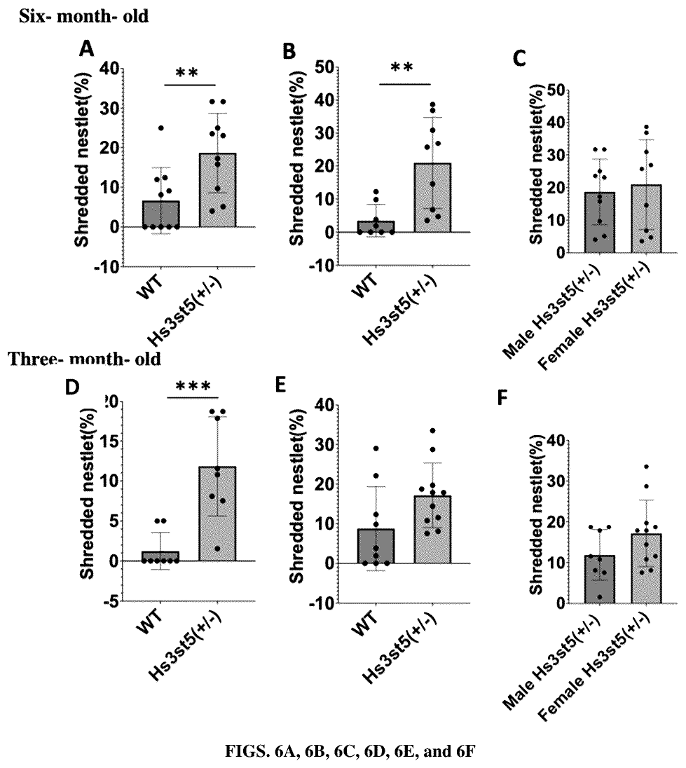

Hs3st4+/− mice: Spatial memory was assessed using the Morris Water Maze (MWM) task, which consists of a water-filled pool with a hidden platform at a fixed location. Over four consecutive days, the mice underwent training sessions, each consisting of four 60-second trials. During each trial, escape latency, and the distance traveled to reach the hidden platform were recorded as measures of acquisition memory. On the fifth day, the percentage of time spent in the target quadrant was measured to assess memory retention without the hidden platform. As shown in FIGS. 1A and 1B, 6-month-old Hs3st4(+/−) male mice spent more time and covered more distance to reach the hidden platform (p<0.01 and p<0.001, respectively). Additionally, there was a significant difference in memory retention (p<0.05) in 3-month-old male Hs3st4(+/−) mice compared to WT mice. Furthermore, 3-month-old female Hs3st4(+/−) mice took more time to find the hidden platform (p<0.001). No significant sex differences in learning and memory were observed between WT and Hs3st4(+/−) mice, except in memory retention among 6-month-old mice, where KO female mice spent less time in the target quadrant compared to male mice (p<0.05).

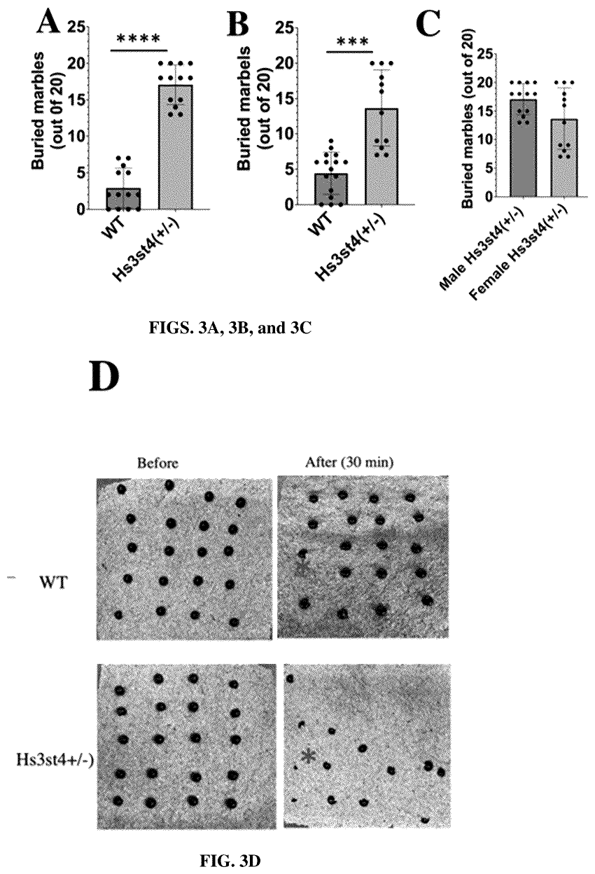

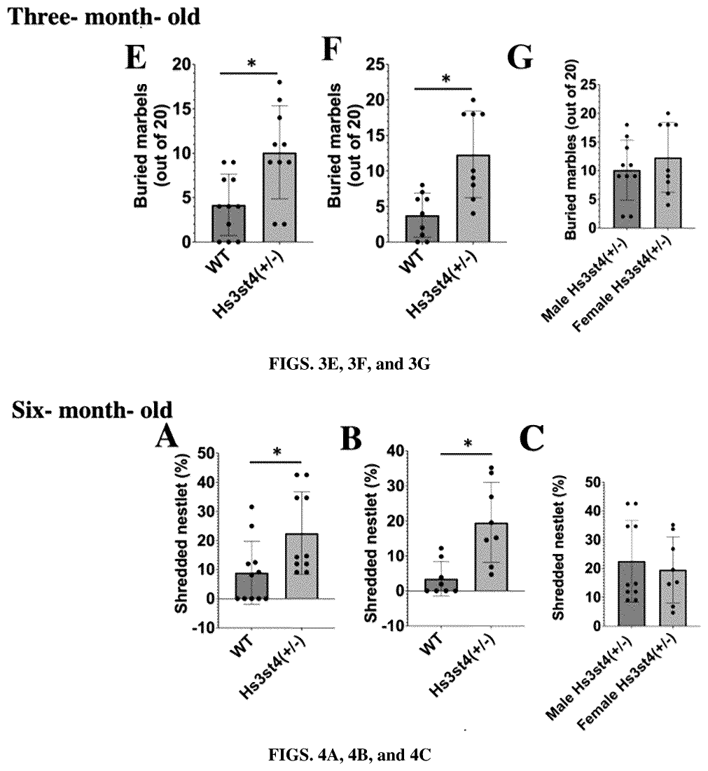

Marble burying and nestlet shredding tests to assess repetitive, compulsive-like behaviors in mice. In the marble burying test, 20 marbles were placed on sawdust bedding in a cage, and each mouse was left in the cage for 30 minutes. After this period, the mouse was returned to its home cage, and the number of buried marbles was counted. A marble was considered buried if two-thirds of its surface was covered by bedding (FIG. 3D). The results showed that 6-month-old male (p<0.0001) and female (p<0.001) Hs3st4(+/−) mice buried more marbles compared to WT mice (FIGS. 3A and B). Additionally, 3-month-old male and female KO mice buried significantly more marbles (p<0.05) compared to WT mice. Further analysis showed no sex-related differences (FIG. 3C).

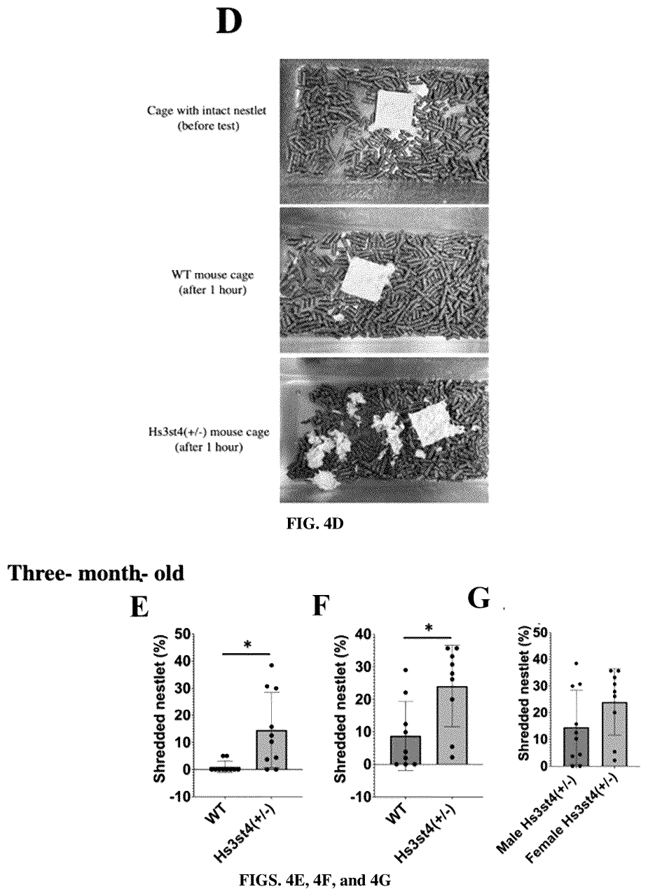

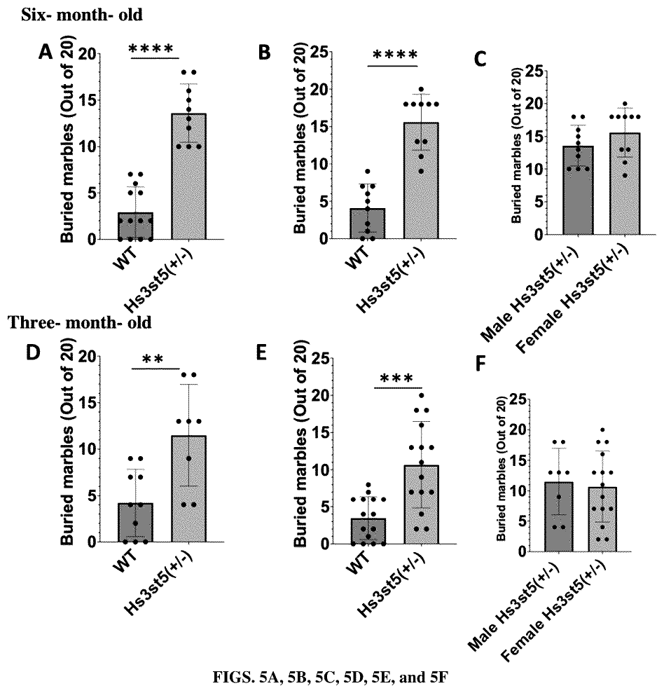

In the nestlet shredding test, a mouse was placed in a cage containing a single, pre-weighed, intact cotton nestlet without access to water and food. After one hour, the remaining intact nestlet was removed and weighed. The percentage of nestlet shredded was calculated by dividing the weight of the unshredded nestlet by the starting weight. As shown in FIGS. 4A-4G, 6-month-old and 3-month-old male (FIG. 4A) and female (FIG. 4B) Hs3st4(+/−) mice shredded more nestlet compared to WT mice (p<0.05). There was no significant difference in the percentage of shredded nestlet between the groups (FIG. 4C).

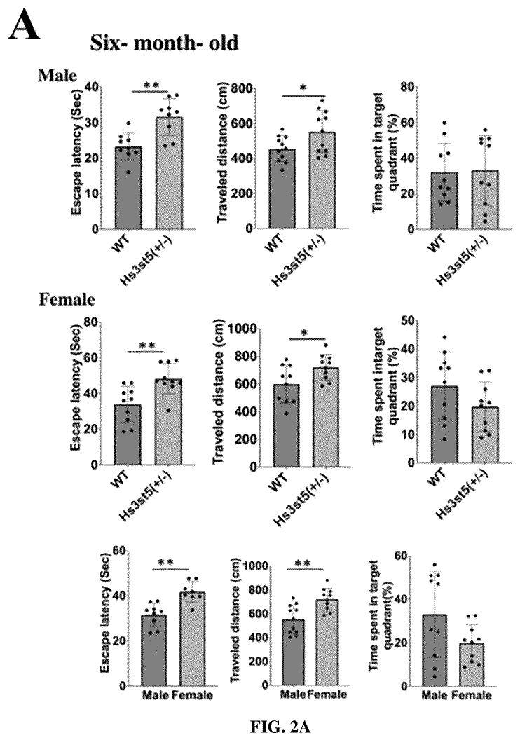

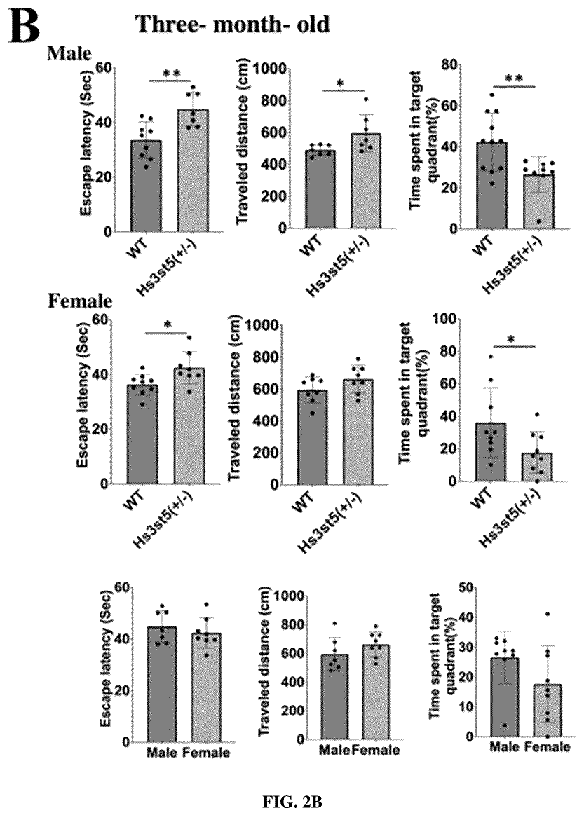

Hs3st5+/− mice: The results of the unpaired t-test (FIGS. 2A and 2B) showed significant differences in escape latency (P<0.01) and distance traveled (P<0.05) in both 6-month-old male and female Hs3st5(+/−) mice compared to WT mice, with a greater impact observed in female mice (P<0.01). Although there was no difference in time spent in the target quadrant among 6-month-old mice, 3-month-old male and female Hs3st5(+/−) mice spent significantly less time in the target quadrant compared to WT mice (p<0.01 and p<0.05, respectively). Additionally, 3-month-old male KO mice traveled a longer path to reach the platform (p<0.05). Escape latency increased in 3-month-old male (p<0.01) and female (p<0.05) Hs3st5(+/−) mice compared to WT mice. No sex-related differences were observed in 3-month-old mice.

The identical marble burying and nestlet shredding protocols were employed to evaluate repetitive, compulsive-like behaviors in Hs3st5(+/−) mice. The results of the marble burying test revealed hyperactivity in Hs3st5(+/−) mice. Both 6-month-old male and female KO mice buried more marbles compared to WT mice (p<0.00001). Additionally, the number of buried marbles was higher in 3-month-old male (p<0.0) and female (p<0.001) Hs3st5(+/−) mice compared to WT mice. There was no sex-related difference in the percentage of buried marbles (FIG. 5C).

As depicted in FIGS. 6A-6F, both 6-month-old male (FIG. 6A) and female (FIG. 6B) Hs3st5(+/−) mice shredded more nestlets compared to WT mice (p<0.01). There was a notable difference in the percentage of shredded nestlet in 3-month-old male Hs3st5(+/−) mice compared to WT mice (p<0.001). No sex-related differences were observed in the weight of unshredded nestlet in either 6-month-old or 3-month-old Hs3st5(+/−) mice (FIG. 6C).

### Histological Studies

The day after memory assessment, 6-month-old male mice (n=4-5 per group) were perfused transcardially with 4% paraformaldehyde, and their brains were coronally sectioned into 50 μm slices using a vibratome. After washing and blocking in 3% normal serum, the sections were incubated overnight with primary antibodies: Iba1 (Thermo Fisher GT10312, 1:1000), CD68 (Bio-Rad, MCA1957GA, 1:100), podocalyxin (R&D, AF1556, 1:400), claudin5 (Invitrogen, WD322260, 1:500), MAPII (Novus, NBP2-25156, 1:1000), synaptophysin (Cell Signaling, D8FH, 1:200), PSD95 (Cell Signaling, D27E11, 1:400), TH (Millipore, AB152, 1:1000), and NeuN (Cell Signaling, 94403, 1:600). This was followed by incubation with ALEXA FLUOR™ secondary antibodies for 1 hour. Fluorescent 4′,6-diamidino-2-phenylindole (DAPI) stain was used as a counterstain. Four to five sections from each brain were stained, and 3-5 different fields from each section were photographed using a Keyence fluorescence microscope.

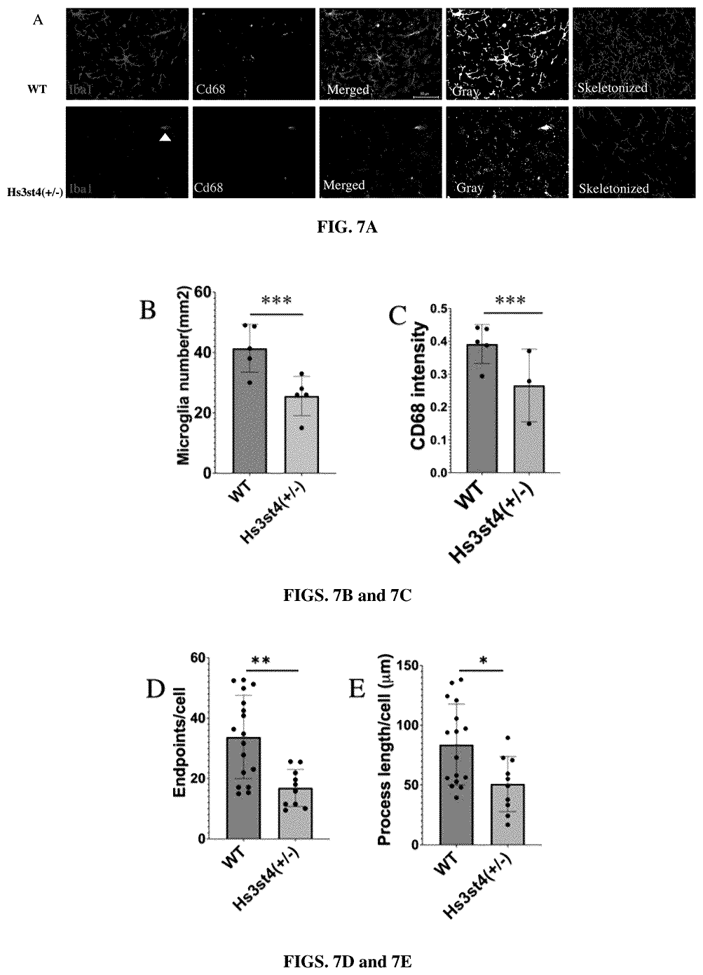

Since Hs3st4 is expressed in microglia, the morphology and function of microglia was assessed in KO and WT mice using double staining for Iba1 and CD68 (FIGS. 7A-7E). The results revealed alterations in the numbers, morphology, and function of microglia in the prefrontal cortex of male Hs3st4(+/−) mice. Immunohistochemical analysis of Iba1 and CD68+ cells in the prefrontal cortex showed ramified surveilling microglia in WT mice, whereas amoeboid microglia were observed in Hs3st4(+/−) mice (FIG. 7A). An unpaired t-test analysis indicated a significant reduction in the number (FIG. 7B) and function (FIG. 7C) of microglia in Hs3st4(+/−) mice. Analysis of skeletonized microglia revealed reduced endpoints (FIG. 7D) and process length (FIG. 7E) in Hs3st4(+/−) mice compared to WT mice.

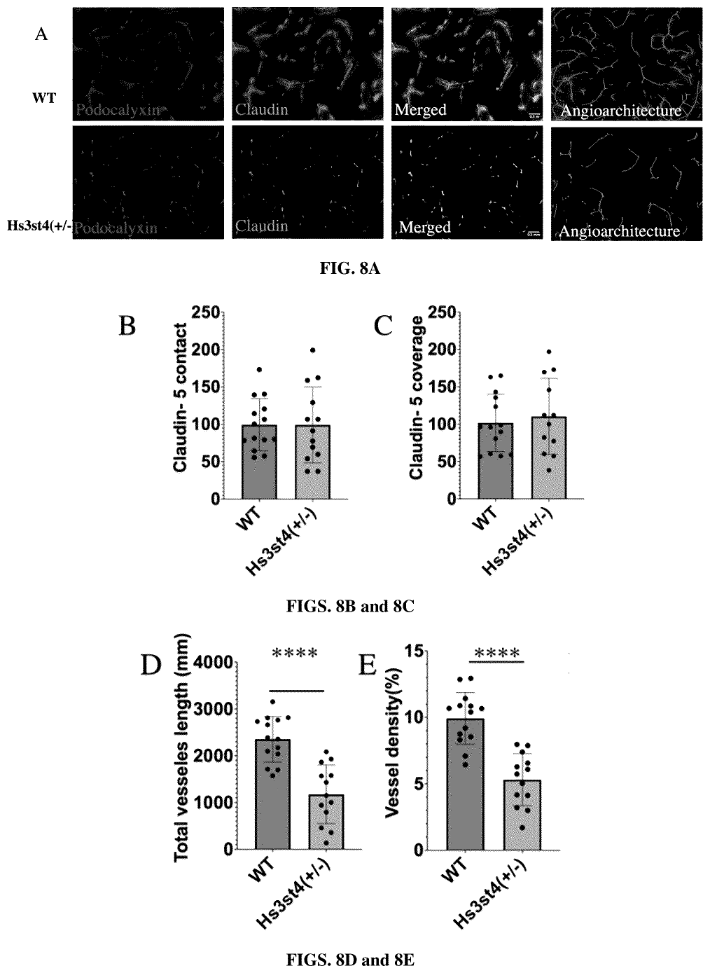

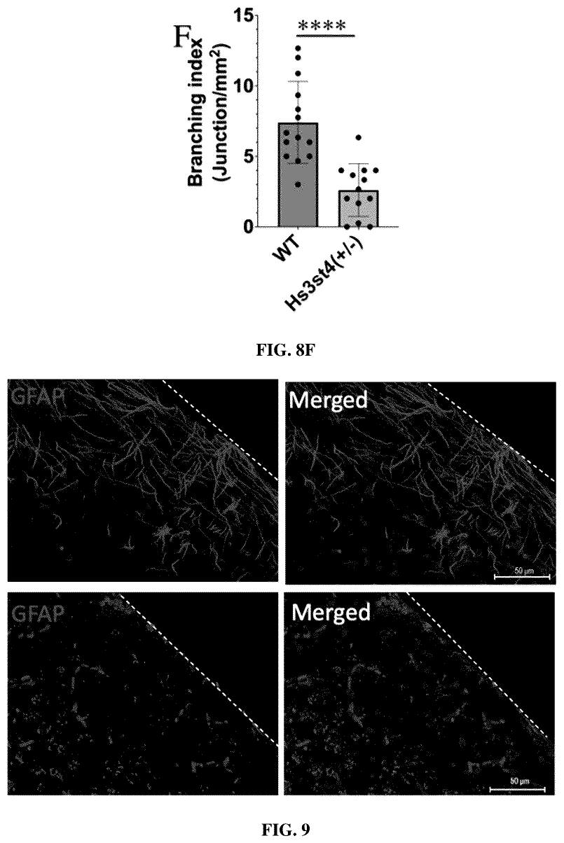

Due to the direct effects of microglia on the blood-brain barrier, the impact of Hs3st4 deficiency was assessed on vascular integrity (FIGS. 8A-8F). Sections were stained with podocalyxin (which is expressed on endothelial cells and promotes adhesion interactions and barrier formation) and claudin (a marker of tight junctions) (FIG. 8A). Quantification of fluorescent intensity using ImageJ software revealed no significant differences in podocalyxin contact (FIG. 8B) and coverage (FIG. 8C), or in claudin contact and coverage in the prefrontal cortex of WT and Hs3st4(+/−) mice.

Subsequently, angioarchitectural analyses were conducted using AngioTool software (FIG. 8A). Remarkably, there was a significant decrease in vessel length (FIG. 8D), vessel density (FIG. 8E), and branching index (FIG. 8F) in Hs3st4(+/−) mice. These findings strongly show that Hs3st4 deficiency markedly affects the structural integrity of cerebral vasculature.

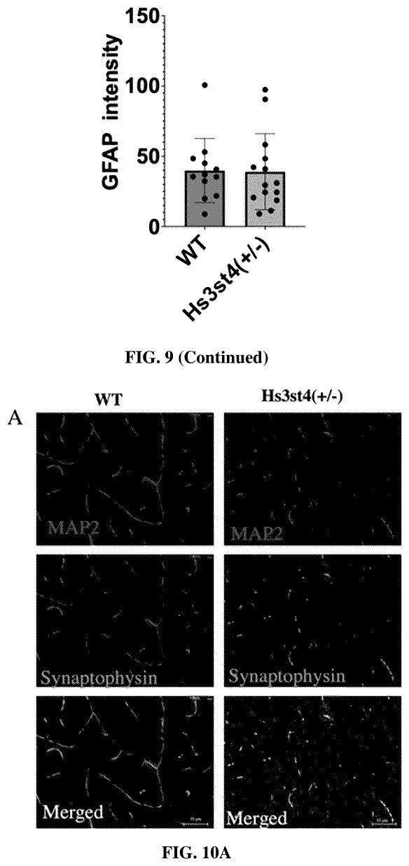

Next, the effect of Hs3st4(+/−) deficiency on the astrocyte phenotype was evaluated. Although immunostaining of the prefrontal cortex revealed an alteration in the distribution of superficial layer cortical astrocytes in 6-month-old male Hs3st4(+/−) mice, there was no significant difference in the intensity of GFAP-positive cells (FIG. 9).

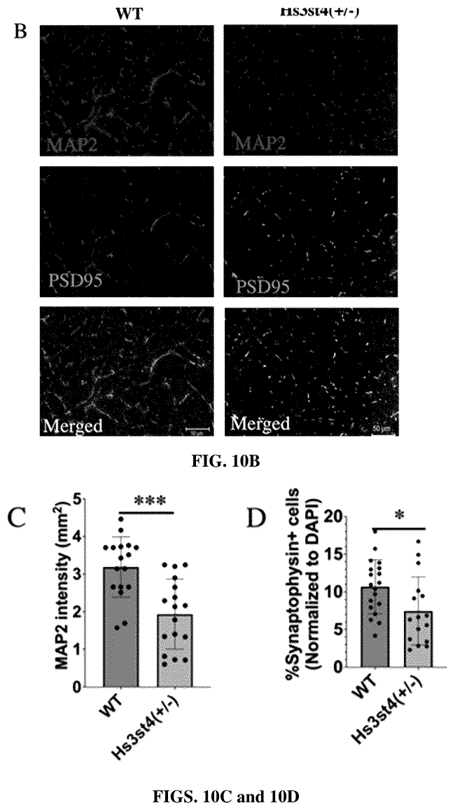

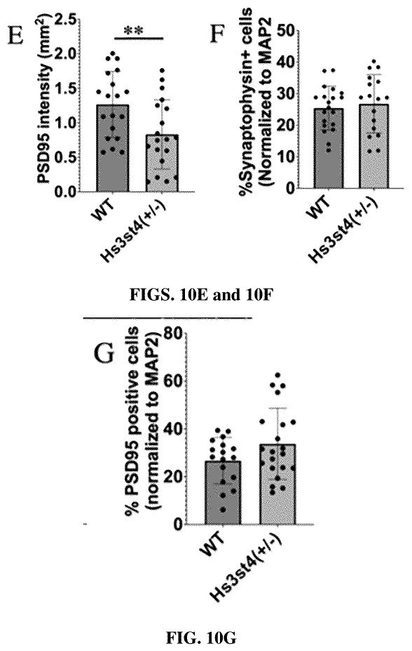

To evaluate the impact of Hs3st4 deletion on synaptogenesis, the prefrontal cortex of WT and Hs3st4(+/−) male mice was stained with MAPII (mature neuron marker), Synaptophysin (presynaptic neuron marker), and PSD95 (postsynaptic neuron marker) (FIGS. 10A and 10B). Analysis using an unpaired t-test revealed a significant reduction in the intensity of MAP2 (FIG. 10C), Synaptophysin (FIG. 10D), and PSD95 (FIG. 10E) in Hs3st4(+/−) mice. However, no significant differences were observed between the groups when the intensities of Synaptophysin (FIG. 10F) or PSD95 (FIG. 10G) were normalized to MAP2.

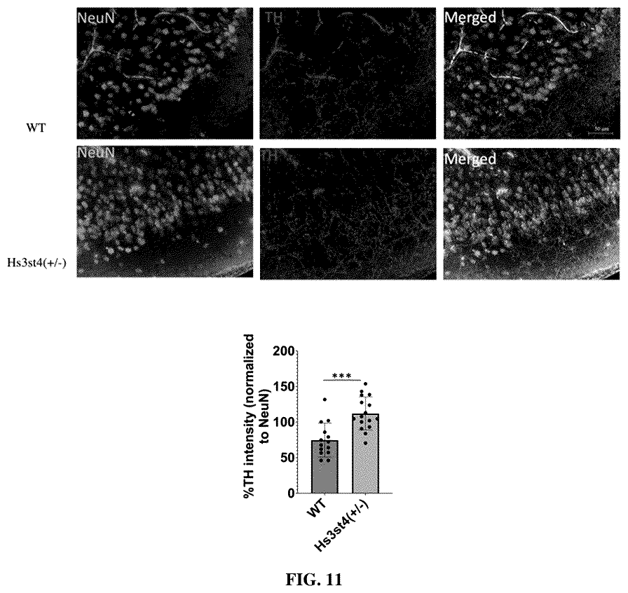

Tyrosine hydroxylase (TH) is the rate-limiting enzyme in dopamine synthesis pathway. Since the Hs3st4(+/−) mice showed ADHD-related phenotype, we assessed the expression of TH in the prefrontal cortex of male mice (FIG. 11). The results showed a remarkable increase in the expression on TH in the prefrontal cortex of Hs3st4(+/−) mice when compared to WT mice (p<0.001).

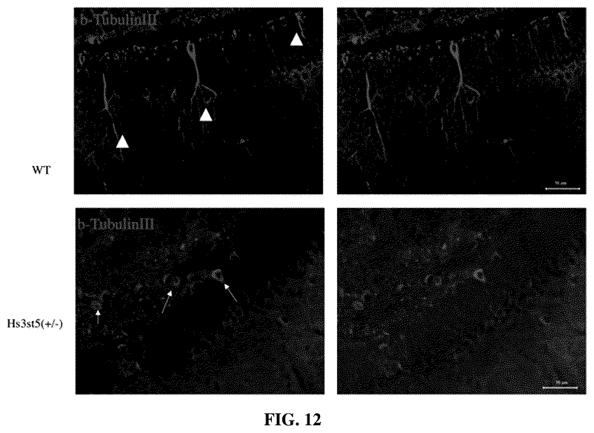

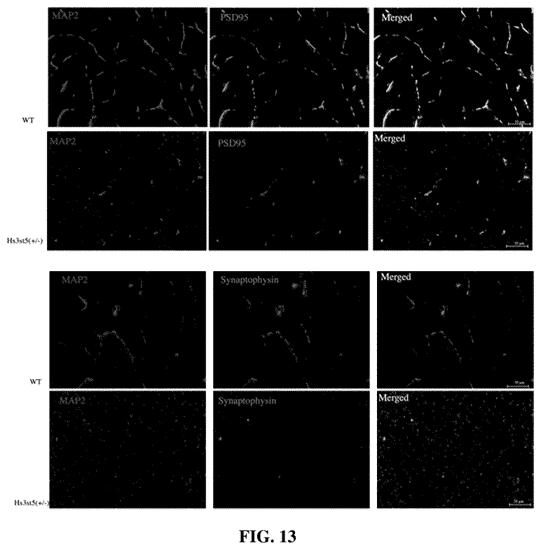

The animals histologically phenotyped after initial characterization of the Hs3st5(+/−) mice. First, the dentate gyrus of the hippocampi was stained with β Tubulin III and differences in the morphology of positive cells between WT and Hs3st5(+/−) mice was observed. Neurons in WT mice exhibited a normal multipolar morphology with distinct fibers, whereas neurons in Hs3st5(+/−) mice appeared apolar or with smaller fibers (FIG. 12). Next, phenotyping of the cortex in both groups using MAPII, Synaptophysin, and PSD95 antibodies revealed heterogeneity in the morphology and distribution of synapses that warrant further evaluation (FIG. 13).

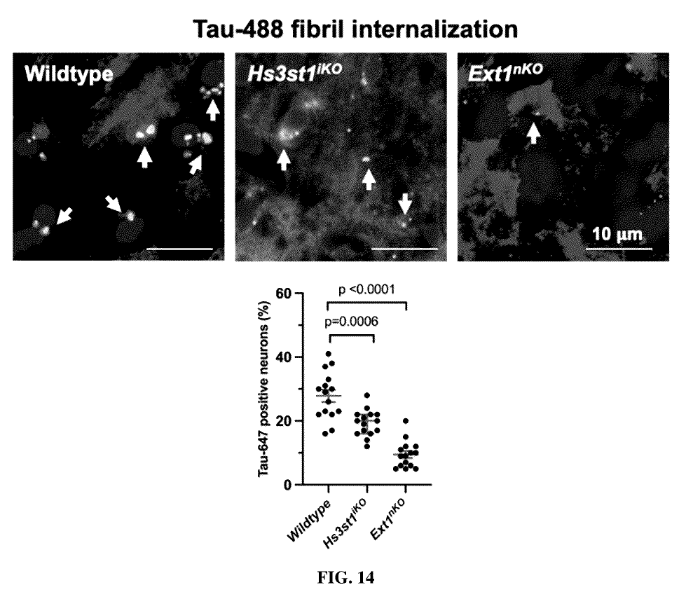

To investigate the potential role of Hs3st1 in Tau propagation in the mouse brain, Tau-488 fibrils (0.5 μg) were injected into the hippocampus of 6-month-old male WT, Cre(+)Hs3st1(KO), or Emx1Cre+Ext1(KO) mice (n=3 each group) at coordinates AP: +1.3 mm, ML: +1.5 mm, DV: −1.6 mm. Brains were sectioned two days after injection and immunostained with an Anti-MAP2 antibody to label neurons. The findings indicate a significant reduction in Tau uptake by cortical neurons in Hs3st1 knockout mice compared to WT mice (FIG. 14).

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
Hs3st1 Targeting Design Summary

| Item | Description |
|---|---|
| Targeting strategies presented | 1 |
| Floxed Exons | Exon 2 |
| sgRNA(s) insertion sites | Intron 1-2 and downstream of Exon 2 |
| Total floxed size | ~2.5 kb |
| Elements in use | 2 loxP |
| Targeting strategy method | See FIG. 15A |
| Gene targeting method | CRISPR/Cas9 based EGE |

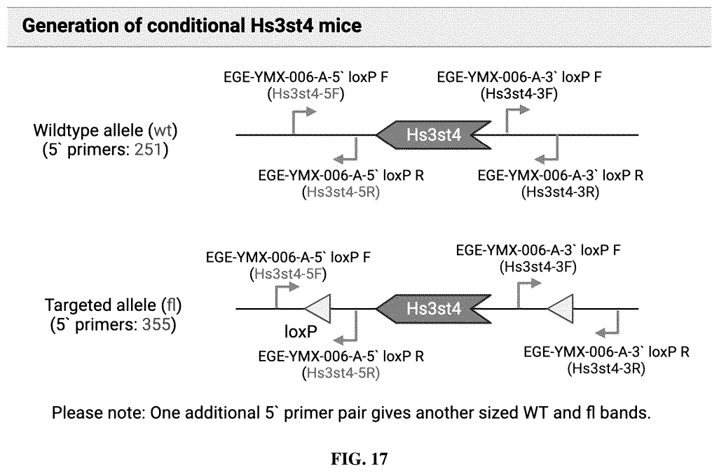

TABLE 2
Hs3st4 Targeting Design Summary

| Item | Description |
|---|---|
| Targeting strategies presented | 1 |
| Floxed Exons | Exon 2 |
| sgRNA(s) insertion sites | Intron 1-2 and downstream of Exon 2 |
| Total floxed size | ~3.5 kb |
| Elements in use | 2 loxP |
| Targeting strategy method | See FIG. 17 |
| Gene targeting method | CRISPR/Cas9 based EGE |

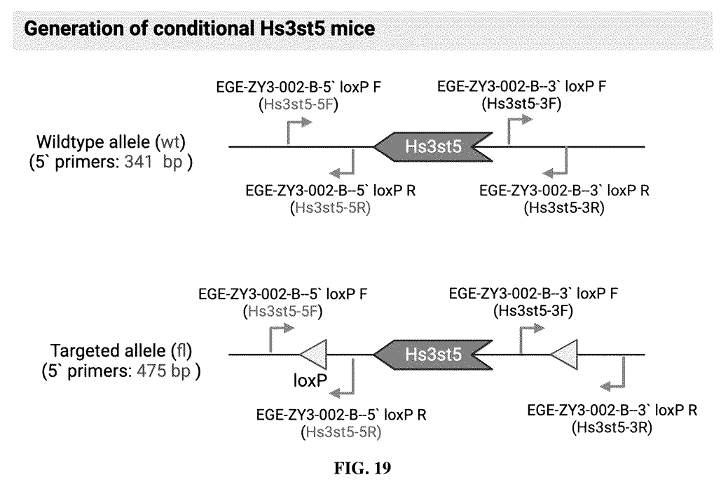

TABLE 3
Hs3st5 Targeting Design Summary

| Item | Description |
|---|---|
| Targeting strategies presented | 1 |
| Floxed Exons | Exon 6 |
| sgRNA(s) insertion sites | Intron 5-6 and downstream of Exon 6 |
| Total floxed size | ~3.0 kb |
| Elements in use | 2 loxP |
| Targeting strategy method | See FIG. 19 |
| Gene targeting method | CRISPR/Cas9 based EGE |

TABLE 4
Conditional Hs3st and iCreERT2 mouse ID

| Generation Code | Genotype |
|---|---|
| EGE-YQH-075 | floxed Hs3st1 |
| EGE-YMX-006 | floxed Hs3st4 |
| EGE-ZY3-002 | floxed Hs3st5 |
| B-CAG-iCreERT2 | Tamoxifen inducible Cre ubiquitous expression |

TABLE 5
EGE-YQH-075-B (Hst3st1) Before Cre-loxP recombination

| | | | Product |
|---|---|---|---|
| | | Tm | size |
| Primer | Sequence (5′-3′) | (° C.) | (bp) |
| EGE-YQH-075-B- | GGACGGAATCATAGGCTTCTTGCCT | 62 | WT: 363 |
| 5′loxP-F (Hs3st1-5F) | (SEQ ID NO: 1) | | FL: 457 |
| EGE-YQH-075-B- | CCTCACCTCCCTCAAACGCTCTATT | 61 | |
| 5′loxP-R (Hs3st41-5R) | (SEQ ID NO: 2) | | |
| EGE-YQH-075-B- | GGCTGGTCACAAAAGCCAGTTCTAAG | 61 | WT: 562 |
| 3′loxP-F (Hs3st1-3F) | (SEQ ID NO: 3) | | FL: 647 |
| EGE-YQH-075-B- | GCACCAGGAAAAACATGTCAGGTCAA | 61 | |
| 3′loxP-R (Hs3st1-3R) | (SEQ ID NO: 4) | | |

TABLE 6
EGE-YQH-075-B (Hst3st1) After Cre-loxP recombination

| | | | Product |
|---|---|---|---|
| | | Tm | size |
| Primer | Sequence (5′-3′) | (° C.) | (bp) |
| EGE-YQH-075-B- | GGACGGAATCATAGGCTTCTTGCCT | 62 | WT: 363 |
| 5′loxP-F (Hs3st1-5F) | (SEQ ID NO: 5) | | |
| EGE-YQH-075-B- | CCTCACCTCCCTCAAACGCTCTATT | 61 | |
| 5′loxP-R (Hs3st41-5R) | (SEQ ID NO: 6) | | |
| EGE-YQH-075-B- | GGACGGAATCATAGGCTTCTTGCCT | 62 | Deletion |
| 5′loxP-F (Hs3st1-5F) | (SEQ ID NO: 7) | | (−): 493 |
| EGE-YQH-075-B- | GCACCAGGAAAAACATGTCAGGTCAA | 61 | |
| 3′loxP-R (Hs3st41-5R) | (SEQ ID NO: 8) | | |

TABLE 7
EGE-YMX-006-A (Hs3st4) Before Cre-loxP recombination

| | | | Product |
|---|---|---|---|
| | | Tm | size |
| Primer | Sequence (5′-3′) | (° C.) | (bp) |
| EGE-YMX-006- | AATCAGGGGTCAGAAAACCGCCTTA | 61 | WT: 251 |
| A-5′loxP-F2 | (SEQ ID NO: 9) | | FL: 355 |
| EGE-YMX-006- | GGCTGTGGGAAAAAGCCCAAATGAT | 61 | |
| A-5′loxP-R2 | (SEQ ID NO: 10) | | |
| (Hs3st4-5R) | | | |
| EGE-YMX-006- | GTCACCCAATGAGGCACTGGCTAAA | 62 | WT: 505 |
| A-3′loxP-F2 | (SEQ ID NO: 11) | | FL: 590 |
| (Hs3st4-3F) | | | |
| EGE-YMX-006- | GCCATGAGTGGGTTAAGCTCAAGGA | 62 | |
| A-3′loxP-R2 | (SEQ ID NO: 12) | | |
| (Hs3st4-3R) | | | |

TABLE 8
EGE-YMX-006-A (Hs3st4) After Cre-loxP recombination

| | | Tm | Product size |
|---|---|---|---|
| Primer | Sequence (5′-3′) | (° C.) | (bp) |
| EGE-YMX- | AATCAGGGGTCAGAAAACCGCCTTA | 61 | WT: 251 |
| 006-A-5′loxP- | (SEQ ID NO: 13) | | |
| F2 (Hs3st4-5F) | | | |
| EGE-YMX- | GGCTGTGGGAAAAAGCCCAAATGAT | 61 | |
| 006-A-5′loxP- | (SEQ ID NO: 14) | | |
| R2 (Hs3st4-5R) | | | |
| EGE-YMX- | AATCAGGGGTCAGAAAACCGCCTTA | 61 | Deletion: |
| 006-A-5′loxP- | (SEQ ID NO: 15) | | 334 (−) |
| F2 (Hs3st4-5F) | | | |
| EGE-YMX- | GCCATGAGTGGGTTAAGCTCAAGGA | 62 | |
| 006-A-3′loxP- | (SEQ ID NO: 16) | | |
| R2(Hs3st4-5R) | | | |

TABLE 9
EGE-ZY3-002-B (Hs3st5) Before Cre-loxP recombination

| | | | Product |
|---|---|---|---|
| | | Tm | size |
| Primer | Sequence (5′-3′) | (° C.) | (bp) |
| EGE-ZY3-002-B- | GTTCCTACAATCAGCACGATGAAAGTG | 60 | WT: 341 |
| 5′loxP-F (Hs3st5-5F) | (SEQ ID NO: 17) | | FL: 475 |
| EGE-ZY3-002-B- | GGATGCTGAAGGGCAAAGAGGAGG | 63 | |
| 5′loxP-R (Hs3st5-5R) | (SEQ ID NO: 18) | | |
| EGE-ZY3-002-B- | AAGCGATCAAACCACACAGCATTGG | 63 | WT: 458 |
| 3′loxP-F (Hs3st5-3F) | (SEQ ID NO: 19) | | FL: 543 |
| EGE-ZY3-002-B- | TGTTGCTGCTCTGCCTGTGTCATTCTG | 64 | |
| 3′loxP-R (Hs3st5-3R) | (SEQ ID NO: 20) | | |

TABLE 10
EGE-ZY3-002-B (Hs3st5) After Cre-loxP recombination

| | | | Product |
|---|---|---|---|
| | | Tm | size |
| Primer | Sequence (5′-3′) | (° C.) | (bp) |
| EGE-ZY3-002- | GTTCCTACAATCAGCACGATGAAAGTG | 60 | WT: 341 |
| B-5′loxP-F | (SEQ ID NO: 21) | | |
| (Hs3st5-5F) | | | |
| EGE-ZY3-002- | GGATGCTGAAGGGCAAAGAGGAGG | 63 | |
| B-5′loxP-R | (SEQ ID NO: 22) | | |
| (Hs3st5-5R) | | | |
| EGE-ZY3-002- | GTTCCTACAATCAGCACGATGAAAGTG | 60 | Deletion |
| B-5′loxP-F | (SEQ ID NO: 23) | | (-): 463 |
| (Hs3st5-5F) | | | |
| EGE-ZY3-002- | TGTTGCTGCTCTGCCTGTGTCATTCTG | 64 | |
| B-3′loxP-R | (SEQ ID NO: 24) | | |
| (Hs3st5-5R) | | | |

TABLE 11
Primer sets and predicted product sizes. Mut: representing the
mutant/targeted allele.

| | | | Product |
|---|---|---|---|
| | | Tm | size |
| Primer | Sequence (5′-3′) | (° C.) | (bp) |
| B-EM-045-WT- | AGTCGCTCTGAGTTGTTATCAG | 56 | WT: 469 |
| F | (SEQ ID NO: 25) | | Mut: 7103 |
| B-EM-045-WT- | TGAGCATGTCTTTAATCTACCTCGATG | 58 | |
| R | (SEQ ID NO: 26) | | |
| B-EM-045-Mut- | CTCTATGACCTGCTGCTGGAG | 58 | Mut: 341 |
| F | (SEQ ID NO: 27) | | |
| B-EM-045-Mut- | ACGGGAAGCAATAGCATGATAC | 57 | |
| R | (SEQ ID NO: 28) | | |

TABLE 12
Hs3st1, Hs3st4, and Hs3st5 Genotyping PCR reaction system

| Reaction Component | Volume (μL) | Final Concentration | |
|---|---|---|---|
| ddH₂O | 6.9 | — | |
| 2X Taq Plus Master Mix II | 10 | 1X | |
| (Dye Plus) | | | |
| 10 mM Primer F | 0.8 | 0.4 | mM |
| 10 mM Primer R | 0.8 | 0.4 | mM |
| 100-200 ng/μL Template | 1.5 | 7.5-15 | ng/μL |

DNA

TABLE 13
Hs3st1, Hs3st4, and Hs3st5 Genotyping Thermal cycling program

| Step | Temperature | Time | | Cycles |
|---|---|---|---|---|
| 1 | 95° C. | 3 | min | 1 |
| 2 | 95° C. | 15 | sec | 32 |
| 3 | 62° C. | 20 | sec | |
| 4 | 72° C. | 1 | kb/min | |
| 5 | 72° C. | 7 | min | 1 |

| | | | |
|---|---|---|---|
| 6 | 4° C. | hold | 1 |

TABLE 14
Hs3st1 Corresponding Genotypes

| EGE-YQH-075-B-5’loxP-F/ | | EGE-YQH-075-B-3’loxP-F/ | | |
|---|---|---|---|---|
| EGE-YQH-075-B-5’loxP-R | | EGE-YQH-075-B-3’loxP-R | | |
| WT | Mut | WT | Mut | Genotype |
| N | Y | N | Y | fl/fl |
| Y | Y | Y | Y | fl/+ |
| Y | N | Y | N | +/+ |

N: No PCR product detected with gel electrophoresis
Y: PCR product detected with gel electrophoresis
fl/fl: representing homozygote genotype
fl/+: representing heterozygote genotype
+/+: representing wild-type genotype

TABLE 15
Hs3st1 Corresponding Genotypes

| | |
|---|---|
| B-EM-045-Mut-F/ | B-EM-045-WT-F/ |
| B-EM-045-Mut-R | B-EM-045-WT-R |

| Mut | WT | Mut | Genotype |
|---|---|---|---|
| Y | N | N* | Mut/Mut |
| Y | Y | N* | Mut/+ |
| N | Y | N | +/+ |

N: No PCR product detected with gel electrophoresis
Y: PCR product detected with gel electrophoresis
*when the primer set B-EM-045-WT-F/B-EM-045-WT-R is used for PCR, the product of targeted allele could be amplified, but sometimes this PCR product is too long to be amplified, so the here is labels as “N*”.
Mut/Mut: representing homozygous genotype
Mut/+: representing heterozygous genotype
+/+: representing wild-type genotype

TABLE 16
Hs3st1 Genotyping results

| Number | Animal ID | Genotype |
|---|---|---|
| 1 | 3SE6N75CAG-0019 | EGE-YQH-075-B(fl/fl); |
| | | CAG-iCre-ERT2(+/+) |
| 2 | 3SE6N75CAG-0020 | EGE-YQH-075-B(fl/+); |
| | | CAG-iCre-ERT2(Mut/+) |
| 3 | 3SE6N75CAG-0021 | EGE-YQH-075-B(fl/fl); |
| | | CAG-iCreERT2(+/+) |
| 4 | 3SE6N75CAG-0023 | EGE-YQH-075-B(fl/+); |
| | | CAG-iCreERT2(Mut/+) |

TABLE 17
Hs3st4 Genotyping Results

| Number | Animal ID | Genotype |
|---|---|---|
| 1 | 4SEN6CAG-0008 | fl/+; CAG-iCreERT2(Mut/+) |
| 2 | 4SEN6CAG-0009 | fl/+; CAG-iCreERT2(Mut/+) |
| 3 | 4SEN6CAG-0010 | fl/fl; CAG-iCreERT2(+/+) |

TABLE 18
Hs3st5 Genotyping Results

| Number | Animal ID | Genotype |
|---|---|---|
| 1 | 3SE14Y2B-0019 | fl/fl |
| 2 | 3SE14Y2B-0022 | fl/fl |
| 3 | 4SE14Y2BC-0018 | EGE-ZY3-002-B(fl/+); |
| | | CAG-iCreERT2(Mut/+) |
| 4 | 4SE14Y2BC-0021 | EGE-ZY3-002-B(fl/+); |
| | | CAG-iCreERT2(Mut/+) |

### Sequences

1. -EGE-YQH-075-B-5′loxP-F (Hs3st1-5F)
SEQ ID NO: 1
GGACGGAATCATAGGCTTCTTGCCT
2. -EGE-YQH-075-B-5′loxP-R (Hs3st41-5R)
SEQ ID NO: 2
CCTCACCTCCCTCAAACGCTCTATT
3. -EGE-YQH-075-B-3′loxP-F (Hs3st1-3F)
SEQ ID NO: 3
GGCTGGTCACAAAAGCCAGTTCTAAG
4. -EGE-YQH-075-B-3′loxP-R (Hs3st1-3R)
SEQ ID NO: 4
GCACCAGGAAAAACATGTCAGGTCAA
5. -EGE-YQH-075-B-5′loxP-F (Hs3st1-5F)
SEQ ID NO: 5
GGACGGAATCATAGGCTTCTTGCCT
6. -EGE-YQH-075-B-5′loxP-R (Hs3st41-5R)
SEQ ID NO: 6
CCTCACCTCCCTCAAACGCTCTATT
7. -EGE-YQH-075-B-5′loxP-F (Hs3st1-5F)
SEQ ID NO: 7
GGACGGAATCATAGGCTTCTTGCCT
8. -EGE-YQH-075-B-3′loxP-R (Hs3st41-5R)
SEQ ID NO: 8
GCACCAGGAAAAACATGTCAGGTCAA
9. -EGE-YMX-006-A-5′loxP-F2 (Hs3st4-5F)
SEQ ID NO: 9
AATCAGGGGTCAGAAAACCGCCTTA
10. -EGE-YMX-006-A-5′loxP-R2 (Hs3st4-5R)
SEQ ID NO: 10
GGCTGTGGGAAAAAGCCCAAATGAT
11. -EGE-YMX-006-A-3′loxP-F2 (Hs3st4-3F)
SEQ ID NO: 11
GTCACCCAATGAGGCACTGGCTAAA
12. -EGE-YMX-006-A-3′loxP-R2 (Hs3st4-3R)
SEQ ID NO: 12
GCCATGAGTGGGTTAAGCTCAAGGA
13. -EGE-YMX-006-A-5′loxP-F2 (Hs3st4-5F)
SEQ ID NO: 13
AATCAGGGGTCAGAAAACCGCCTTA
14. -EGE-YMX-006-A-5′loxP-R2 (Hs3st4-5R)
SEQ ID NO: 14
GGCTGTGGGAAAAAGCCCAAATGAT
15. -EGE-YMX-006-A-5′loxP-F2 (Hs3st4-5F)
SEQ ID NO: 15
AATCAGGGGTCAGAAAACCGCCTTA
16. -EGE-YMX-006-A-3′loxP-R2 (Hs3st4-5R)
SEQ ID NO: 16
GCCATGAGTGGGTTAAGCTCAAGGA
17. -EGE-ZY3-002-B-5′loxP-F (Hs3st5-5F)
SEQ ID NO: 17
GTTCCTACAATCAGCACGATGAAAGTG
18. -EGE-ZY3-002-B-5′loxP-R (Hs3st5-5R)
SEQ ID NO: 18
GGATGCTGAAGGGCAAAGAGGAGG
19. -EGE-ZY3-002-B-3′loxP-F (Hs3st5-3F)
SEQ ID NO: 19
AAGCGATCAAACCACACAGCATTGG
20. -EGE-ZY3-002-B-3′loxP-R (Hs3st5-3R)
SEQ ID NO: 20
TGTTGCTGCTCTGCCTGTGTCATTCTG
21. -EGE-ZY3-002-B-5′loxP-F (Hs3st5-5F)
SEQ ID NO: 21
GTTCCTACAATCAGCACGATGAAAGTG
22. -EGE-ZY3-002-B-5′loxP-R (Hs3st5-5R)
SEQ ID NO: 22
GGATGCTGAAGGGCAAAGAGGAGG
23. -EGE-ZY3-002-B-5′loxP-F (Hs3st5-5F)
SEQ ID NO: 23
GTTCCTACAATCAGCACGATGAAAGTG
24. -EGE-ZY3-002-B-3′loxP-R (Hs3st5-5R)
SEQ ID NO: 24
TGTTGCTGCTCTGCCTGTGTCATTCTG
25. -B-EM-045-WT-F
SEQ ID NO: 25
AGTCGCTCTGAGTTGTTATCAG
26. -B-EM-045-WT-R
SEQ ID NO: 26
TGAGCATGTCTTTAATCTACCTCGATG
27. -B-EM-045-Mut-F
SEQ ID NO: 27
CTCTATGACCTGCTGCTGGAG
28. -B-EM-045-Mut-R
SEQ ID NO: 28
ACGGGAAGCAATAGCATGATAC
29. -EGE-YMX-006-A-5′loxP-F
SEQ ID NO: 29
AAATCAGGGGTCAGAAAACCGCCTT
30. -EGE-YMX-006-A-5′loxP-R
SEQ ID NO: 30
TGGCAAATTCTCCTTGCCTCCTGTT

What is claimed is:

1. A transgenic mouse comprising a nucleic acid sequence comprising a heterologous heparan sulfate-glucosamine 3-sulfotransferase (HS3ST) gene and at least two loxP nucleic acid sequences, and wherein a native HS3ST gene is deleted in the transgenic mouse.

2. The transgenic mouse of claim 1, wherein the native HS3ST gene or the heterologous HS3ST gene comprises HS3ST1, HS3ST4, or HS3ST5.

3. The transgenic mouse of claim 1, further comprising a transgene encoding a Cre recombinase.

4. The transgenic mouse of claim 3, wherein the Cre recombinase is an inducible Cre recombinase or a conditional Cre recombinase.

5. A method of generating a transgenic HS3ST mouse, the method comprising:
a. targeting a native HS3ST gene within a mouse with one or more single guide ribonucleic acid (sgRNA) sequences and a CRISPR-Cas9 system, wherein the CRISPR-Cas9 system excises the native HS3ST gene, and
b. inserting a nucleic acid sequence to replace the native HS3ST gene, wherein the nucleic acid sequence comprises a heterologous HS3ST gene and at least two loxP nucleic acid sequences.

6. The method of claim 5, further comprising breeding the transgenic HS3ST mouse with a Cre recombinase transgenic mouse to generate an HS3ST x Cre transgenic mouse.

7. The method of claim 5, wherein the transgenic HS3ST mouse further comprises a nucleic acid sequence encoding a Cre recombinase.

8. The method of claim 5, wherein the native HS3ST gene or the heterologous HS3ST gene comprises HS3ST1, HS3ST4, or HS3ST5.

9. The method of claim 5, wherein the method excises a native HS3ST1 gene or a native HS3ST4 gene by targeting the nucleic acid sequence upstream of intron 1 and intron 2 of exon 2, and the nucleic acid sequence downstream of the native HS3ST1 gene or the native HS3ST4 gene.

10. The method of claim 5, wherein the method of excises a native HS3ST5 gene by targeting the nucleic acid sequence upstream of intron 5 and intron 6 of exon 6, and the nucleic acid sequence downstream of the native HS3ST5 gene.

11. The method of claim 7, wherein the Cre recombinase is an inducible Cre recombinase or conditional Cre recombinase.

12. The method of claim 7, wherein the Cre recombinase excises the heterologous HS3ST gene.

13. The method of claim 6, wherein the HS3ST x Cre mouse comprises a conditional deletion of the heterologous HS3ST gene.

14. The method of claim 13, wherein the conditional deletion comprises a cell-specific deletion, a tissue-specific deletion, a developmental-specific deletion, a disease-stage specific deletion, or a combination thereof.

15. A method of screening for a therapeutic agent for treating or preventing a heparan sulfate-related disease, the method comprising:
a. breeding a heparan sulfate-glucosamine 3-sulfotransferase (HS3ST) transgenic mouse with a Cre recombinase transgenic mouse to generate an HS3ST x Cre transgenic mouse, wherein the HS3ST transgenic mouse and the HS3ST x Cre transgenic mouse comprise a nucleic acid sequence comprising a heterologous HS3ST gene and at least two loxP nucleic acid sequences;
b. activating expression of a Cre recombinase within the HS3ST x Cre transgenic mouse, wherein the Cre recombinase excises the heterologous HS3ST gene;
c. inducing one or more phenotypes in the HS3ST x Cre transgenic mouse;
d. administering the therapeutic agent to the HS3ST x Cre transgenic mouse; and
e. detecting an improvement of the one or more phenotypes in the HS3ST x Cre transgenic mouse relative to an untreated transgenic mouse.

16. The method of claim 15, wherein the HS3ST gene comprises HS3ST1, HS3ST4, or HS3ST5.

17. The method of claim 15, wherein the Cre recombinase is an inducible Cre recombinase or conditional Cre recombinase.

18. The method of claim 15, wherein the Cre recombinase excises the heterologous HS3ST gene.

19. The method of claim 15, wherein the one or more phenotypes are caused by a conditional deletion of the heterologous HS3ST gene.

20. The method of claim 19, wherein the conditional deletion comprises a cell-specific deletion, a tissue-specific deletion, a developmental-specific deletion, a disease-stage specific deletion, or a combination thereof.
